(19) United States
(12) Patent Application Publication
(10) Pub. No.: US 2016/0229362 A1
(43) Pub. Date: Aug. 11, 2016

(54) Auxiliary power system called a D C Powerbox

(71) Applicants: Steven Brutus, Columbus, IN (US); George Griffiths, Southlake, TX (US)

(72) Inventors: Steven Brutus, Columbus, IN (US); George Griffiths, Southlake, TX (US)

(21) Appl. No.: 14/986,503

(22) Filed: Dec. 31, 2015

Related U.S. Application Data
(60) Provisional application No. 62/100,446, filed on Jan. 6, 2015.

(51) Int. Cl. B60R 16/03; B60K 5/08; B60L 1/00; G08B 21/14; H02K 5/00; F25D 23/00; G01N 33/00; B60R 16/033; H02K 7/18

(52) U.S. Cl. CPC B60R 16/0307; B60R 16/033; B60K 5/08; B60L 1/00; H02K 7/1815; H02K 5/00; F25D 23/00; G01N 33/004; G08B 21/14; B60H 1/00414

(57) ABSTRACT

An auxiliary Powerbox to generate electricity through an additional alternator configuration in an emergency vehicle which reduces fuel consumption and minimizes wear factors associated with idling the engine when the vehicle is at rest and when the vehicle's primary engine is not operating. The system is an auxiliary system that provides electrical power by means of a small internal combustion engine and an alternator and is intended to provide electrical power to a vehicle when the main engine of the vehicle is shut down. The system, the DC Powerbox, has an independent fuel system and cooling system however the fuel and cooling systems could be incorporated into the vehicles respected systems.

### CROSS-REFERENCE TO RELATED APPLICATIONS

This application claims the benefit of United States Provisional Patent Application with Ser. No. 62/100,446 filed Jan. 6, 2015 by Steven Brutus and George Griffiths and entitled “An auxiliary power system called a D C Powerbox”.

### FIELD OF INVENTION

This invention relates to an auxiliary engine and alternator system which will provide additional electrical power to a vehicle in lieu of idling the primary engine. The system further relates to a secondary system for operating high loads of a vehicle, emergency equipment, air conditioning units, supplemental heat, parasitic electrical power drains and other electrical accessories. This auxiliary power system called a D C Powerbox is intended to reduce the need for police, emergency, other government and/or private vehicles from needing to idle the main engine; which support the high electrical loads and thus reducing the engine run hours, fuel consumption and exhaust pollution due to excessive idle time. Therefore this is an auxiliary power system for use on transport vehicles powered by internal combustion engines to provide electrical power during periods when the transport vehicle's main engine can or must be shut off.

### BACKGROUND

### Background and Problems Addressed

In July of 2008 the average cost of retail gasoline in the United States reached an all-time high of $4.05 per gallon. This was nearly a 30% increase from the year before when gas was averaging $2.93 per gallon (Energy Information Administration 2008). Thus one of the issues and problems to be solved that initiated a study of how to reduce fuel consumption without impacting police service was the subject of the study conducted by Brain Donaldson (Donaldson 2008). Through surveys of police officers in 24 different city police departments the Donaldson study provided a snap shot of the vehicles used, time idling, miles driven, some of the fuel conservation actions taken, and the willingness of officers to implement other fuel conversation actions.

The Donaldson study highlighted the rising fuel cost coupled with shrinking public safety budgets to create the need for police departments to become innovated of how resources are used, but concluded each department had to choose which options would work best for their agency and the citizens they served. The Donaldson study reported roughly 80% of the police vehicles were Ford Crown Victoria(s), the remainder of the vehicles were similar to the Crown Victoria i.e. large powerful sedans. The need for this type of vehicle is required for police work. A high performance engine enables the police to rapidly respond to critical situations and provides an advantage in high speed pursuits. The size of the vehicle is also driven by requirements of police work equipment such as: computers, long guns, space for two officers (in the case of field training and partnering officers), along with the space for transporting suspects, and prisoners. Lastly, a large powerful vehicle projects an impression of strong authority. The data from the Donaldson study reported that 75% of the police vehicles were driven between 25 to 75 miles during the day, and 85% of the vehicles idled between 1 to 5 hours in the same day. From prorating the raw data from the Donaldson study, and the established performance of the Crown Victoria found that 41% of the fuel used (cost) was used during idling. Lastly the Donaldson study indicated that over 60% of the officers would consider changing driving habits, roughly 25% had no option, and only 15% would not consider changing their driving habits.

An additional operating cost not directly tied to fuel consumption is vehicle wear. As an example, Ford's official position on idling is that for every hour that the vehicle spends idling it is equivalent to 33 miles driven. Therefore, another problem to solve is to minimize the wear factor by minimizing the idle hours of a vehicle caused by the need for electrical energy to support the high electrical loads when the vehicle is stopped or at rest. This is further seen in the standard option of an hour meter on fleet (Crown Victoria, Ford Explorer, etc.), and the sales of used police vehicles will typically include idle hours in addition to the miles driven; the invention by DC Powerbox will reduce the cost, due to idling police vehicles.

In addition to the study, one can also infer that police cars in particular have become extremely familiar and visible as they patrol various areas throughout the country. In addition to the typical police car, other agencies which have similar functions and needs often maintain a police car type vehicle(s) that are used in a patrol function. Other emergency vehicles such as firefighting vehicles and ambulances utilize many similar systems to those employed in typical police cars. In recent years, a number of developing technologies have been employed in police —type vehicles which have greatly increased the complexity, sophistication and electrical power consumption of these familiar vehicles. The systems have evolved from the simple roof mounted flashing light and siren configurations of years past into complex, often computer equipped multifunction type systems. The vehicles of these various agencies generally employ an equipment set which includes an elongated light bar secured to the vehicle roof and supporting: a plurality of flashing and rotating light apparatus, continue to utilize sirens as well as external high powered speakers for communication, have the conventional vehicle lights themselves such as headlights and taillights which are often tied into the emergency systems, many utilize computer apparatus and data link equipment to further enhance the information available to the police officer, include a radio for communication, and have a growing trend which is the addition of video camera and video recording equipment intended to be operated during events and occurrences. In practice, police agencies and other similar agencies produce patrol cars by converting more or less standard automobiles to patrol cars in a process best described as custom installation of emergency and police equipment. This process has proven to be time consuming and costly. In addition, the creation of custom installations for each patrol car renders the maintenance process more costly and specialized thereby increasing the operating costs of the police department or agency.

Thus, while police departments and other agencies continue to require such patrol vehicles or other similarly equipped vehicles. Whilst problems arise in the accrual of hours and warranty costs when associated with the “life-time of the engine” is exceeded. These associated costs include: the cost of conversion, the cost maintenance of converted vehicles, and the safe operation of such vehicles in their use. There remains therefore a need in the art for an alternate/improved power system that meets the high electrical load requirement and reduces the idle time when the engine is running to support these electrical loads. An auxiliary power system called a D C Powerbox for police/emergency vehicles renders installation and customization as well as the maintenance of a more cost effective system in such vehicles while providing a maximum level of security and safety for vehicle occupants during their use in the field.

### PRIOR ART

As far as known there are no other devices or process that perform the function of the auxiliary power system called a D C Powerbox for police/emergency vehicles. A U.S. Pat. No. 4,672,296 issued to Griffin (1987) and entitled “Mobile emergency medical vehicle with auxiliary engine/generator providing AC/DC output” shows a mobile emergency medical vehicle having a main transport engine and a medical equipment, supply and patient compartment or module, with such compartment having an independently operated electrical generating means powered by an auxiliary engine capable of providing all of the electrical power requirements of the compartment including exterior and interior electrical lighting, vacuum generating means, power outlet means, compartment air conditioning, heating and the like. The electrical generating means is capable of supplying both 115 volt AC and 12 volt DC power through a converter to the compartment. It is very complex and bulky as compared to the present D C Powerbox by Brutus et al. Another U.S. Pat. No. 6,048,288 (2000) to Tsujii et al. entitled “Power train system for a vehicle and method for operating same” discloses an engine wherein auxiliary machines are operated by a motor generator where the engine is stopped to reduce electric power consumption. This uses a motor generator as opposed to the Brutus system which complicates the conversion to the DC system. Further, a U.S. Pat. No. 6,796,367 (2004) to Blacquiere, et al. entitled “Vehicle battery charging and air conditioning operating unit” shows and teaches an auxiliary engine generates electricity through an alternator, to charge the vehicle battery and selectively run an electric motor for air conditioning when the vehicle's primary engine is not operating. Operation of the electric motor produces rotation of the drive shaft of the vehicle's air conditioning compressor, the output shaft of the electric motor being connected via a centrifugal clutch, and a drive belt on a pulley integral to the clutch, to a pulley bolted directly to the drive shaft of the compressor. This device shows several extra belt drives compared to the Brutus system.

The U.S. Pat. No. 7,049,707 (2006) to Wurtele entitled “Auxiliary power unit for a diesel powered transport vehicle” discloses an auxiliary power unit (APU) for a transport vehicle powered by an internal combustion engine. An enclosure houses and supports the APU on the transport vehicle. An air-cooled engine within the enclosure directly drives an air-cooled, brushless generator. The generator provides direct, simultaneous AC and DC voltage outputs without requiring an inverter circuit or a converter circuit. Air duct systems within the enclosure separately convey cooling air into, through and out of the air-cooled engine and generator. The air duct cooling and complex control varies greatly from the Brutus system. A U.S. Patent Application 20060107920 to Serkh et al. entitled “Auxiliary power system for a motor” and a U.S. Patent Application 20070130950 to Serkh et al. both disclose auxiliary power systems for a motor vehicle comprising a first engine having an accessory belt drive system comprising a belt and at least one driven pulley, the belt drivable by a driver pulley, the accessory belt drive system further comprising a motive member for driving the accessory belt drive system, the motive member comprises a motor engaged with the accessory belt drive system, a second engine operable to drive an electric power source, the electric power source electrically connected to the motive member, an alternate power source other than the electric power source connected to the motive member, a switch for selecting between the electric power source and the alternate power source, and the motive member, drivable by the electric power source or the alternate power source in order to drive the accessory belt drive system when the first engine is not operating. Both of the Serkh et al systems are very complex and bulky as compared to the present D C Powerbox by Brutus et al.

Another U.S. Pat. No. 7,259,469 (2007) to Brummett et al. entitled “Vehicle auxiliary power unit, assembly, and related methods” demonstrates an auxiliary power unit having components specifically selected such that they form a stand-alone unit that can fit within an auxiliary compartment of a vehicle and deliver heating, cooling, and additional electric power to a truck tractor. It fails to be a simple, yet complete, system shown by Brutus. Another U.S. Pat. No. 7,291,932 (2007) again to Wurtele et al. entitled “Auxiliary power unit for a diesel powered transport vehicle” discloses a system for use in transport vehicles, comprising in combination an auxiliary power unit (APU) for a transport vehicle powered by a diesel engine and having a fuel system, an engine exhaust system and a battery powered electrical system; and a locator unit for use in a tracking and monitoring system for communicating over a communication network with the locator unit and with a remote location the APU, wherein the locator unit is attached to or installed within the APU. Again this is a very complex and bulky system as compared to the present D C Powerbox by Brutus et al. A U.S. Patent Application 20080023965 to Cagliari et al. entitled “Auxiliary power unit for transportation vehicle” provides and teaches an auxiliary power unit for providing power and HVAC to a vehicle. The auxiliary power unit is mounted to the vehicle in a variety of ways, but such that it can be readily moved away from the vehicle for service and maintenance. The power unit has a removable condenser and fan assembly that can be mounted to the power unit or remote from the human occupied portion of the vehicle. This idea also fails to be a simple, yet complete, system shown by Brutus.

U.S. Pat. No. 7,939,952 (2011) to Borghi entitled “Automotive auxiliary power system” shows an auxiliary power system for a vehicle, having an auxiliary internal combustion engine with a starter is described. An auxiliary alternator driven by the auxiliary engine and connected to a battery of the vehicle and a control unit, which is connected to the battery, and activates the starter of the auxiliary engine in the event the voltage of the battery is below a reference value, may be included. It fails to anticipate the Brutus system in many ways. Next, U.S. Pat. No. 8,118,005 to Bradley et al. (2012) and entitled “Auxiliary power units for vehicles” demonstrates and teaches a cooperative operation of an auxiliary power unit having a mechanical power output shaft with a transmission coupled power takeoff operation system of a vehicle is provided by allowing the vehicle's main engine to be cranked by the auxiliary power unit through the power takeoff operation system. It is very complex as compared to the present D C Powerbox by Brutus et al. Finally, U.S. Pat. No. 8,215,422 to Abel (2012) and entitled “Emergency utility vehicle” shows and discusses an off-road emergency vehicle includes an operator compartment with an environmentally controlled atmosphere and wireless communication devices. The modified, extended all-terrain vehicle frame supports an enclosed equipment compartment having a top and sides. The equipment compartment is large enough to enclose a full-length stretcher for someone in need of care in an off-road area. The equipment compartment also contains a bench for an emergency technician and storage. The vehicle includes a secondary power source used to supply power to the lighting system and equipment of the equipment compartment. It is more complex as compared to the present DC Powerbox by Brutus et al.

### SUMMARY OF THE INVENTION

The invention is named DC Powerbox and is an auxiliary system that provides electrical power by means of a small internal combustion engine and an alternator and is intended to provide electrical power to a vehicle when the main engine of the vehicle is shut down. The invention, DC Powerbox, has an independent fuel system and cooling system however the fuel and cooling systems could be incorporated into the vehicles respected systems. The auxiliary system has sufficient power to operate all of the vehicle's electrical devices, with provisions for heating and cooling the vehicle's cabin. The invention can be incorporated as an auxiliary system (mounting on the vehicles the push bumper, on a bracket arrangement attached to the rear of the vehicle, or as a stand-alone remote means deployed in proximity to the vehicle providing the mission requirements) within the vehicle for the existing electrical system, or may also be part of the OEM vehicle design, complementing the main engine.

The DC Powerbox is intended to provide electrical power to a police vehicle in lieu of idling. From background information police vehicles are built with powerful engines in order to respond to emergency situations quickly and are equipped with high electrical output alternator(s) to power all of the electrical components. Police vehicles that are equipped with these high output alternators experience a loss in output when the engine is idling due to elevated temperature under the hood along with a significant energy lost due to the parasitic loads. Compounding to the energy wasted during idling is the depletion of the useful life of the vehicle. The idle time(s) of police vehicles are recorded, and for roughly every hour idling is the equivalent of driving 33 miles. The idle equivalent miles are added to the driven miles for warranty issues. Even with the consequences of idling, idling is essential in providing the necessary electrical power for lighting and electronic equipment. However these consequences of idling are resolved by the invention DC Powerbox.

The preferred embodiment is an auxiliary power system called a D C Powerbox comprised of: (a) an engine with an output drive shaft; (b) an alternator; (c) a means for the engine to mechanically power or drive the alternator; (d) a means for fueling the engine; (e) a means for controlling the fueling of the engine; (f) a means for providing power from the alternator to a battery; and (g) a means to structurally mount the engine/alternator. This also includes means to connect and/or remove the components (c), (d), (e), and (f) together as an auxiliary power system. Alternative embodiments are: The system described as a preferred embodiment wherein the means to structurally mount to an existing police vehicle OEM or packaged system on a push bar, bracket arrangement. The system described as a preferred embodiment wherein the means to structurally mount is a standalone palletized frame. The system described as a preferred embodiment further comprised a means for providing electrical power for existing or complementary HVAC systems or as a remote stand-alone unit providing the same. The system described as a preferred embodiment further comprised of a means of recharging the vehicles primary battery requirements for normal operation as well as any secondary power requirements. The system described as a preferred embodiment wherein the means for fueling the engine is using an economically viable fuel source. The system described as a preferred embodiment further comprised of a means of displaying and monitoring aspects of charge requirements. The system described as a preferred embodiment further comprised of a means of incorporating design aspects for fuel shut off, choke, remote starting, and enabling switches. The means of providing electrical power so the vehicle can operate without using the primary engine to decrease: fuel consumption, emissions, and hours spent idling.

### OBJECTIVE AND BENEFITS

Implementation of this invention will enable various government agencies to save money with regards to fuel costs by incorporating a DC power supply; which will supply the necessary power requirements without unnecessarily idling to operate the emergency vehicles equipment. There exists a need for an efficient, compact APU of minimal complexity that overcomes the aforementioned disadvantages, and is easily integrated into an existing vehicle's electrical system. The installation of this system in conjunction with an auxiliary heating and cooling units, will directly and efficiently provide both AC and DC electrical power for the vehicle's cabin and for battery recharging at a greatly reduced cost. There also exists a need for a system which allows the air conditioner and other accessories to be operated without running the primary engine, and without excessive energy consumption and emissions. Another objective is to have auxiliary power units for use on police and emergency vehicles powered by an auxiliary internal combustion engines to provide electrical power during periods when the transport vehicle's main engine must be shut off. Recognized, therefore, is the need for a compact auxiliary power unit which is generally self-contained and can be housed in an existing or modified auxiliary compartment of the vehicle, and that includes within the auxiliary compartment a generator to provide electric power, to power various electronic components in the vehicle cabin, including but not limited to a heating system, and/or an air-conditioning system. Recognized also is the need for an auxiliary power unit which includes sensors and circuitry to monitor and control the electrical components and power requirements. Some examples of the benefits for such a system are: significant fuel savings, increased in-service life of the vehicle, and lowering harmful emissions.

### DESCRIPTION OF THE DRAWINGS

### Figures

The accompanying drawings, which are incorporated in and constitute a part of this specification, illustrate an embodiment of the system that is preferred. The drawings together with the summary description aforementioned in addition to a detailed description given below serve to explain the principles of an auxiliary power system called a DC Powerbox. It is understood, however, that the auxiliary power system called a D C Powerbox is not limited to only the precise arrangements and instrumentalities shown.

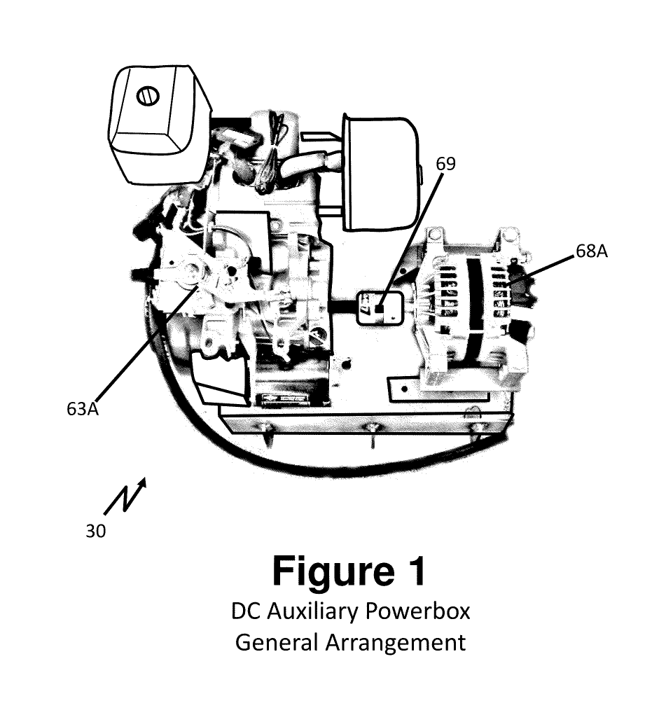

FIG. 1: DC Auxiliary Powerbox General Arrangement.

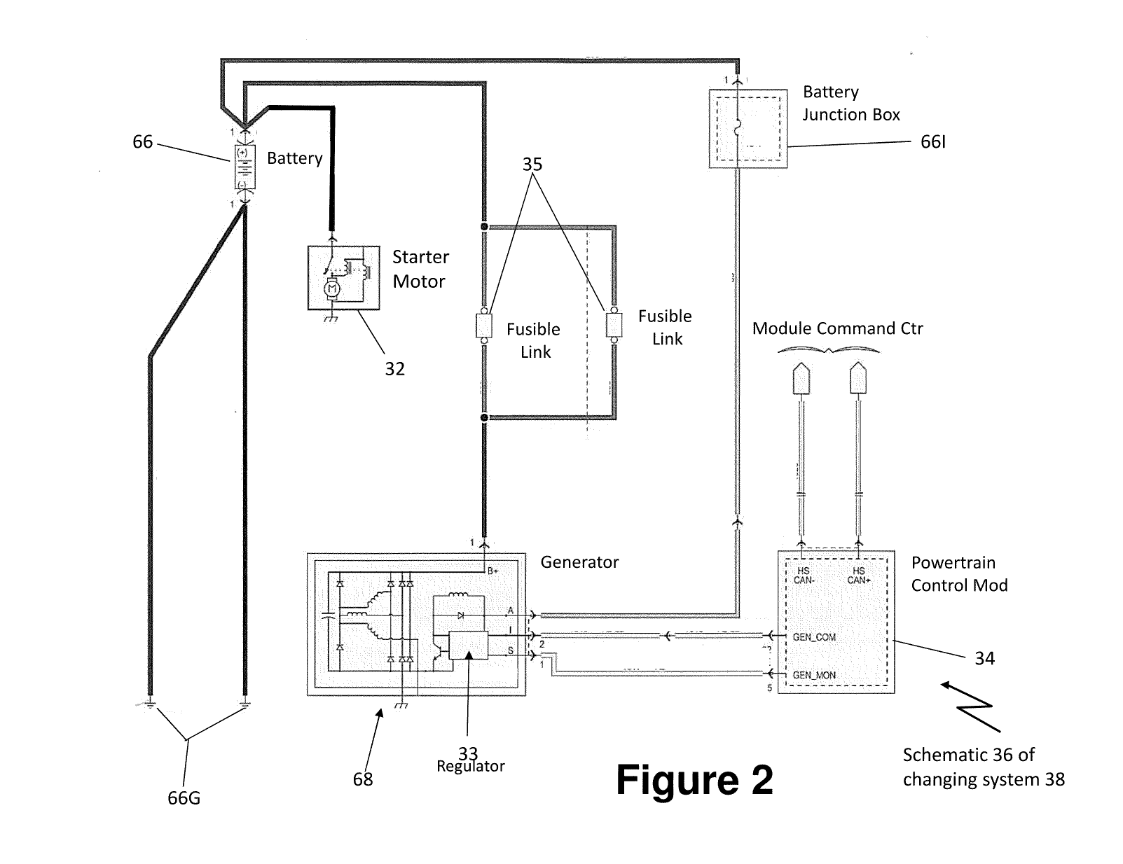

FIG. 2: Example of Vehicle with High Electrical Loads Electrical Charging Schematic.

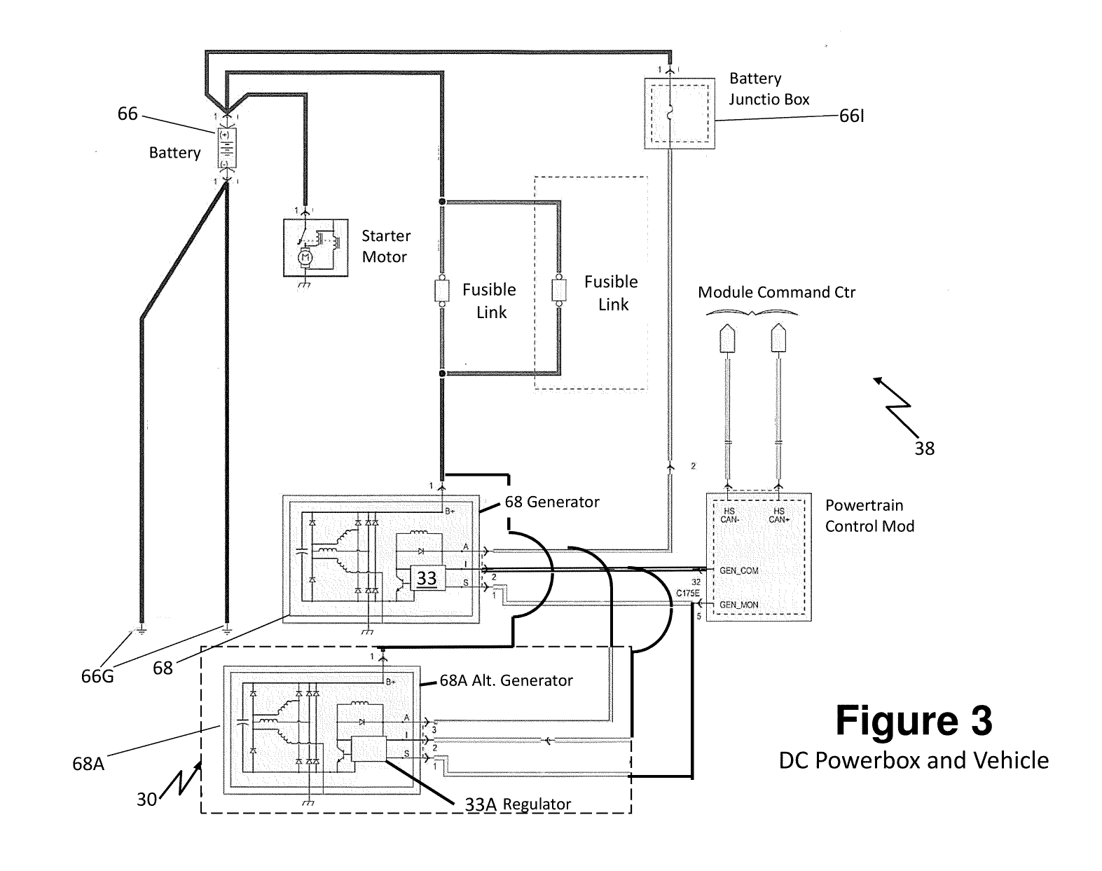

FIG. 3: DC Power box and Vehicle with High Electrical Loads Electrical Charging Schematic.

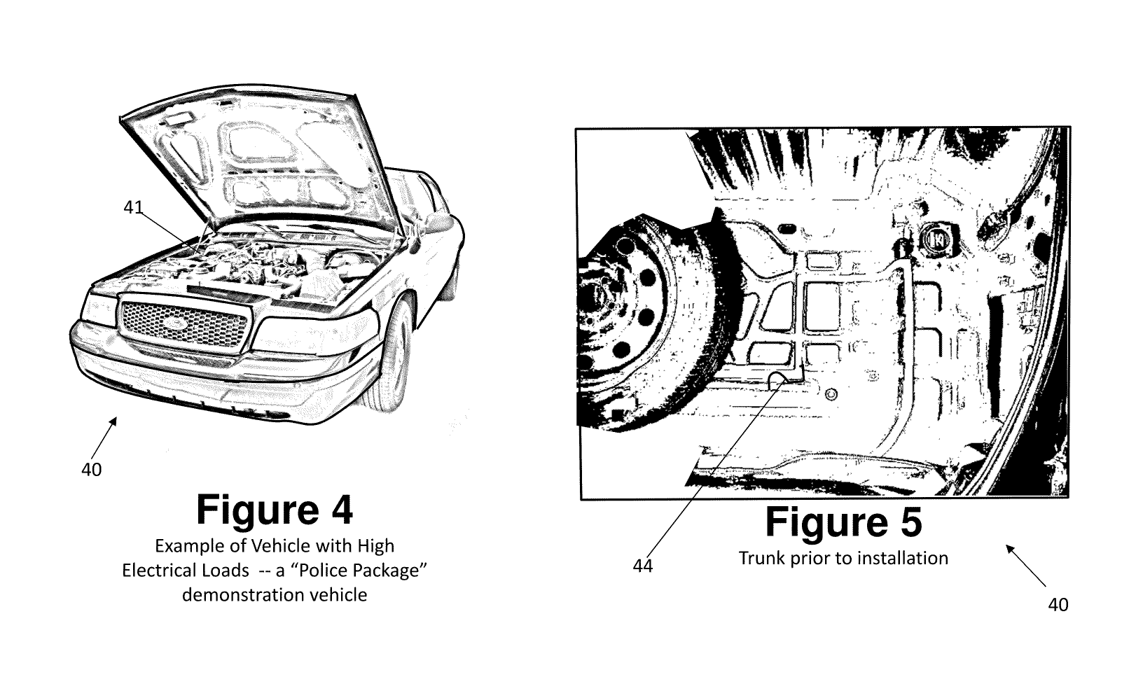

FIG. 4: Vehicle with High Electrical Loads —“Police Package” demonstration vehicle.

FIG. 5: Trunk prior to installation.

FIG. 6: Installed switches front left to right: Master, Fuel, Choke, Start, (Cigarette Lighter), Amp Display

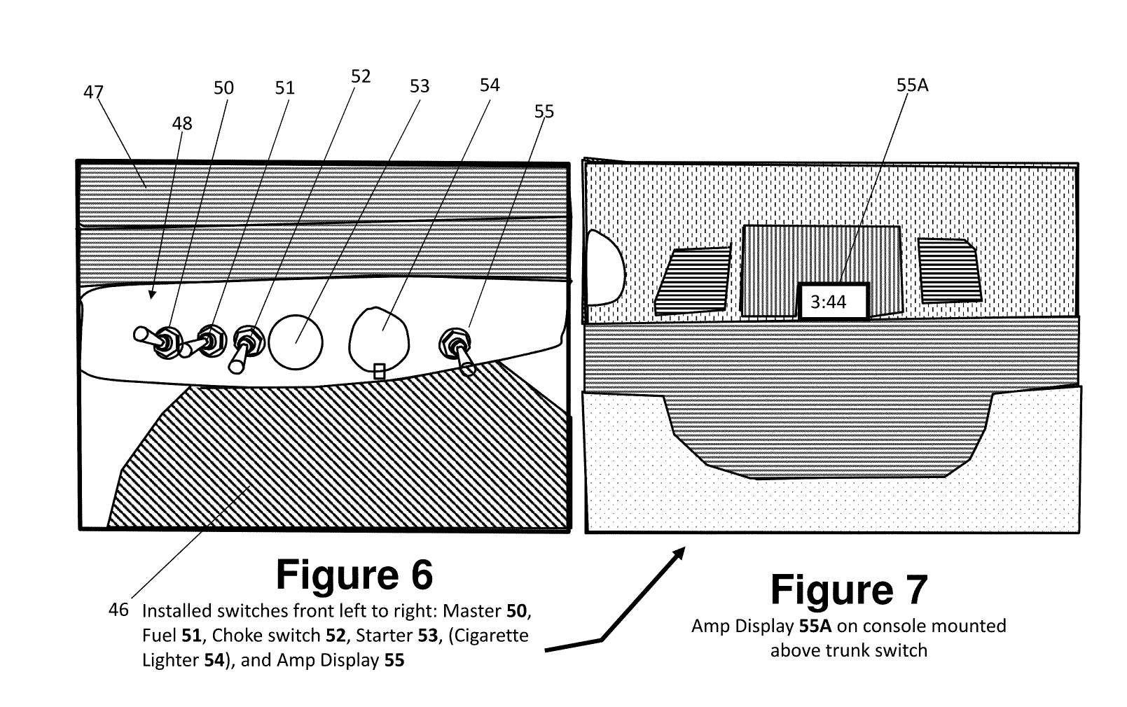

FIG. 7: Amp Display on console mounted above trunk switch—Display: A 3½″ Digital Blue LED DC 200 Amp Current Meter using a 5V (2 Watt) Isolated Power Module for a 12 V system.

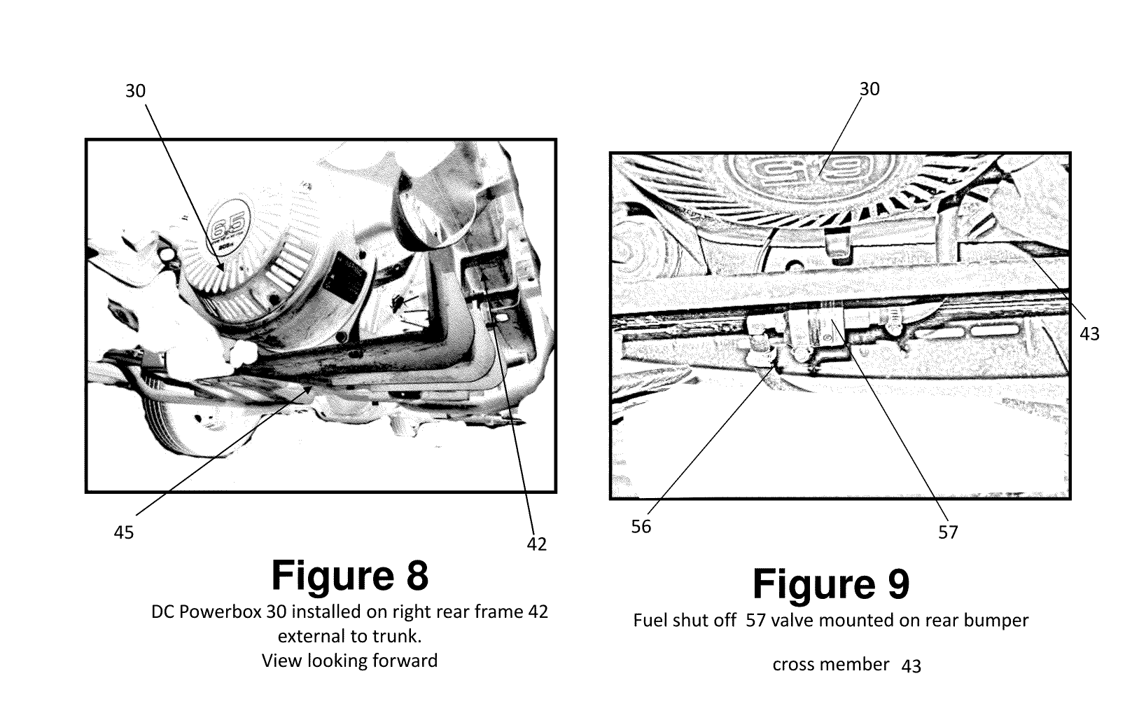

FIG. 8: DC Power box installed on right rear frame external to trunk. View looking forward. Bracket: Steel plate with 2 mounting brackets attached to frame, custom fabricated (insulated) steel box.

FIG. 9: Fuel shut off valve mounted on rear bumper cross member—1 Amp fuel shut off valve (afcvalves.com).

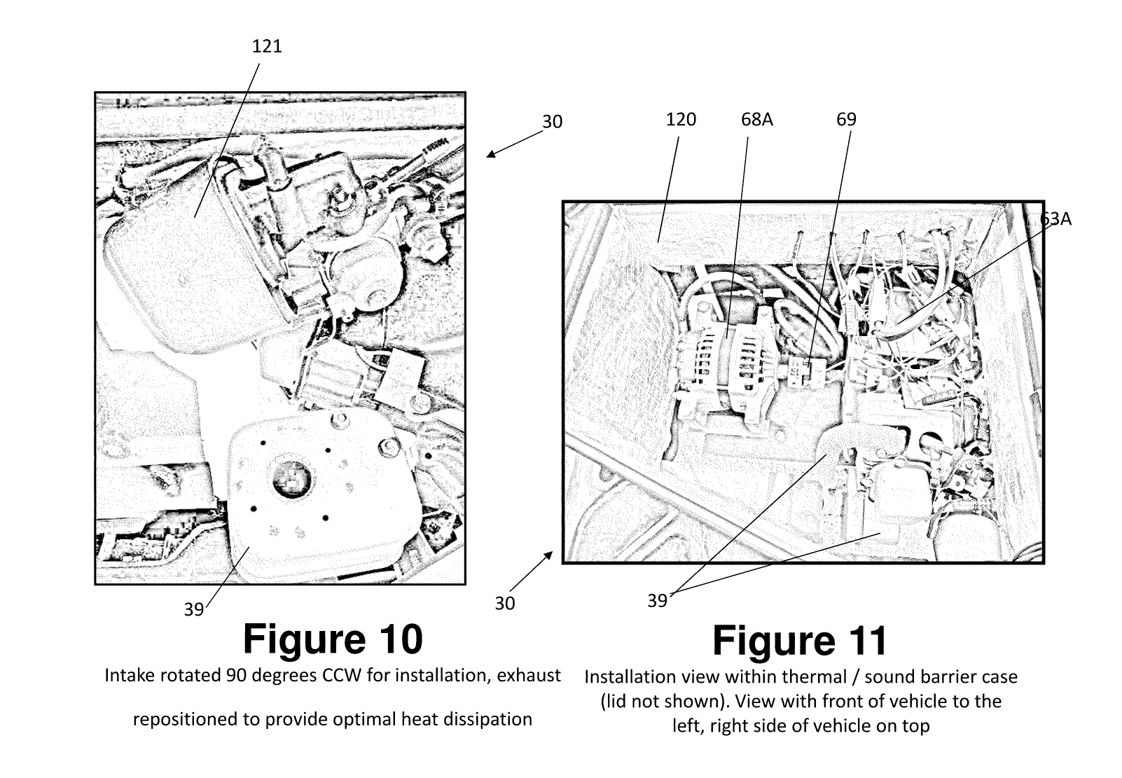

FIG. 10: Intake rotated 90 degrees CCW for installation, exhaust repositioned to provide optimal heat dissipation.

FIG. 11: Installation view within thermal/sound barrier case (lid not shown). View with front of vehicle to the left, right side of vehicle on top.

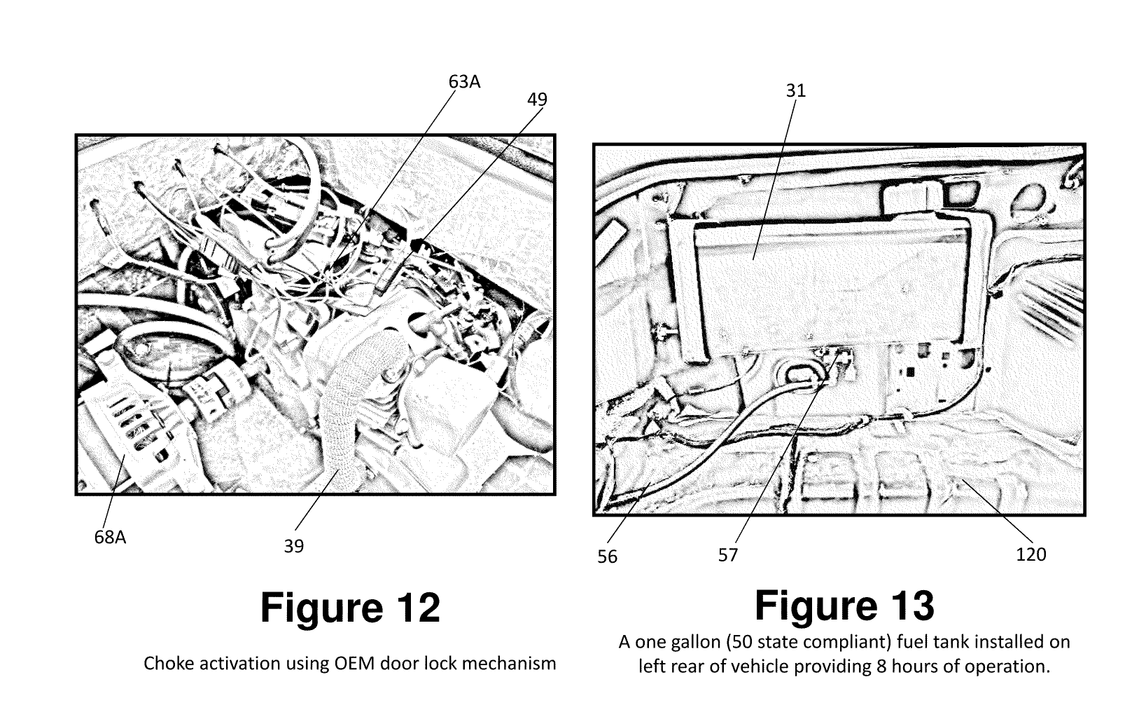

FIG. 12: Choke activation using OEM door lock mechanism.

FIG. 13: A one gallon (50 states compliant) fuel tank installed on left rear of vehicle providing 8 hours of operation. —Tank: Custom Welded 50 state legal cap, brackets, brass fitting (on, off), tubing.

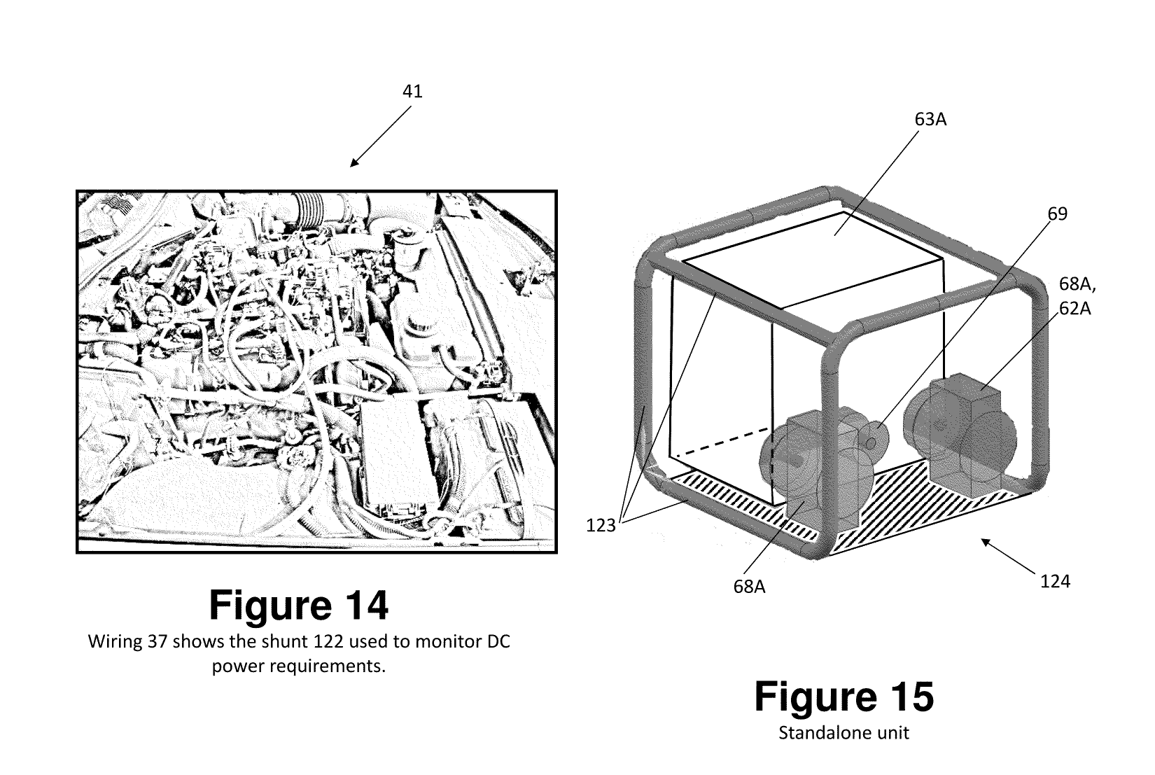

FIG. 14: Wiring that shows shunt used to monitor DC power requirements. Wiring: Master, Fuel (1 amp fuse), Choke (X amp fuse), Start, Hour meter, Power from Battery.

FIG. 15: Standalone unit—Tubing, Plate, Dual Alternators, Pulleys, Belts, Engine, Battery, Wiring.

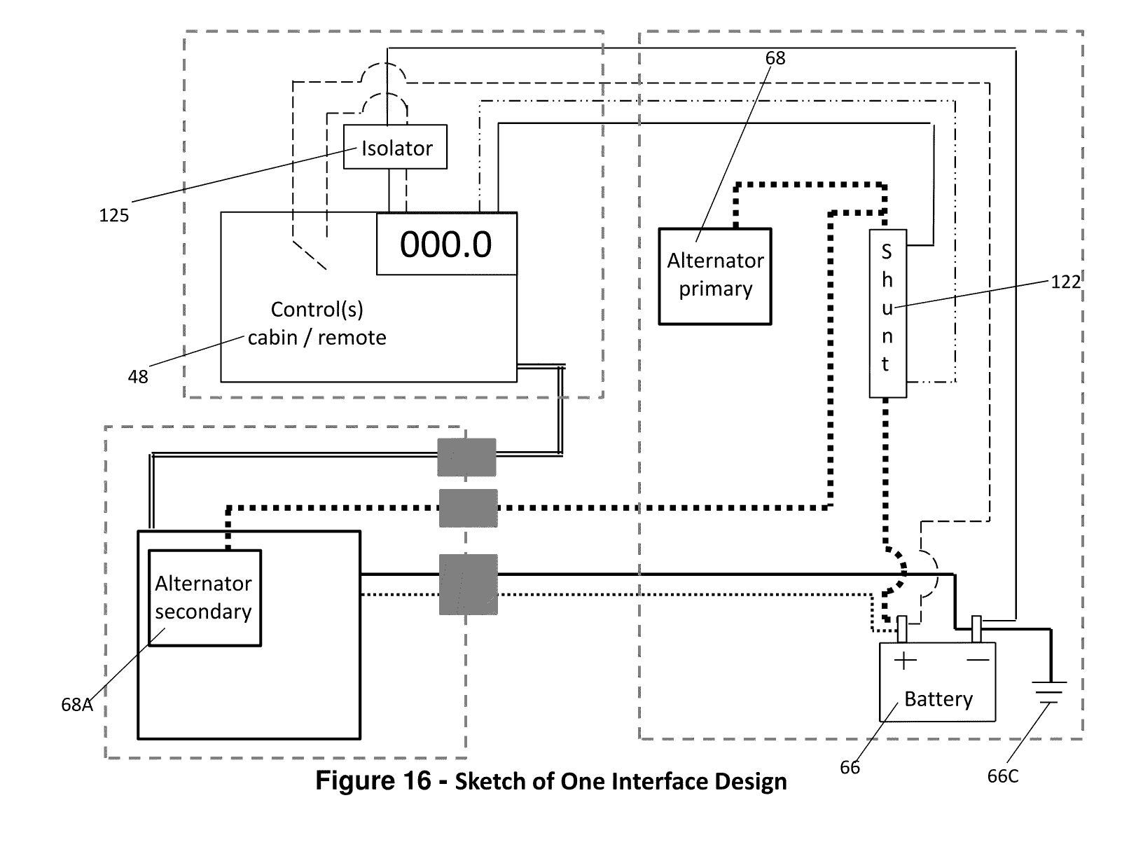

FIG. 16: Sketch of One Interface Design. Wiring: Master, Fuel (1 amp fuse), Choke (X amp fuse), Start, Hour meter, Power from Battery . . . Shunt: 200 Amp 75 mV DC Shunt.

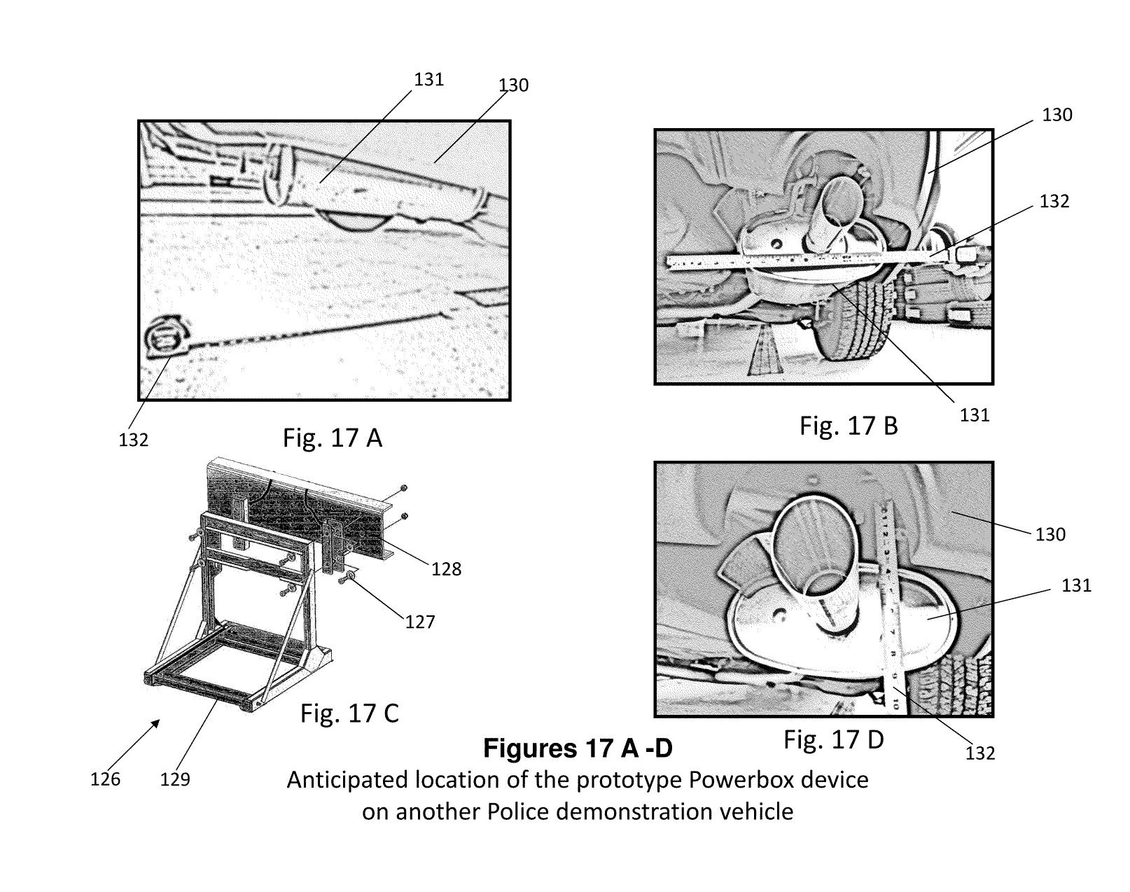

FIGS. 17 A through 17 D: Anticipated location of the Powerbox device on another police demonstration vehicle.

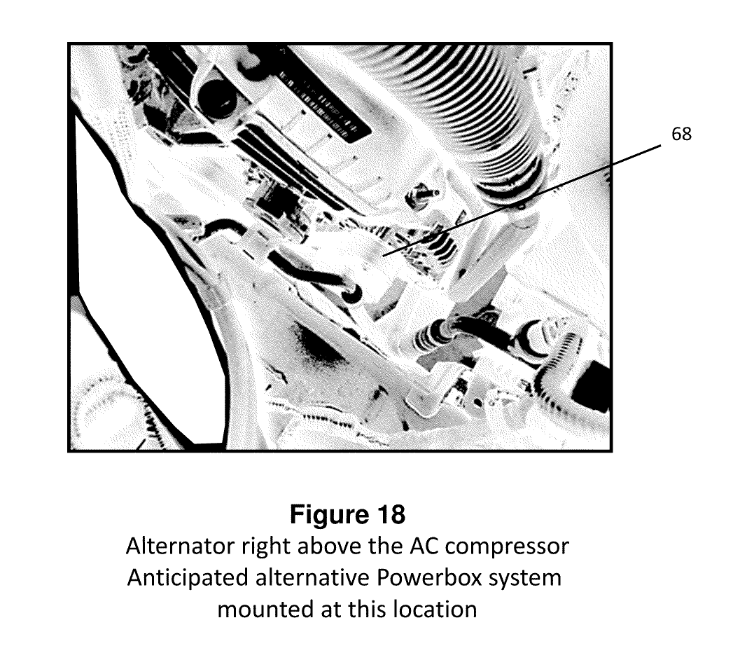

FIG. 18: Alternator right above the AC compressor—it is anticipated as an alternative application of the Powerbox system mounted at this location.

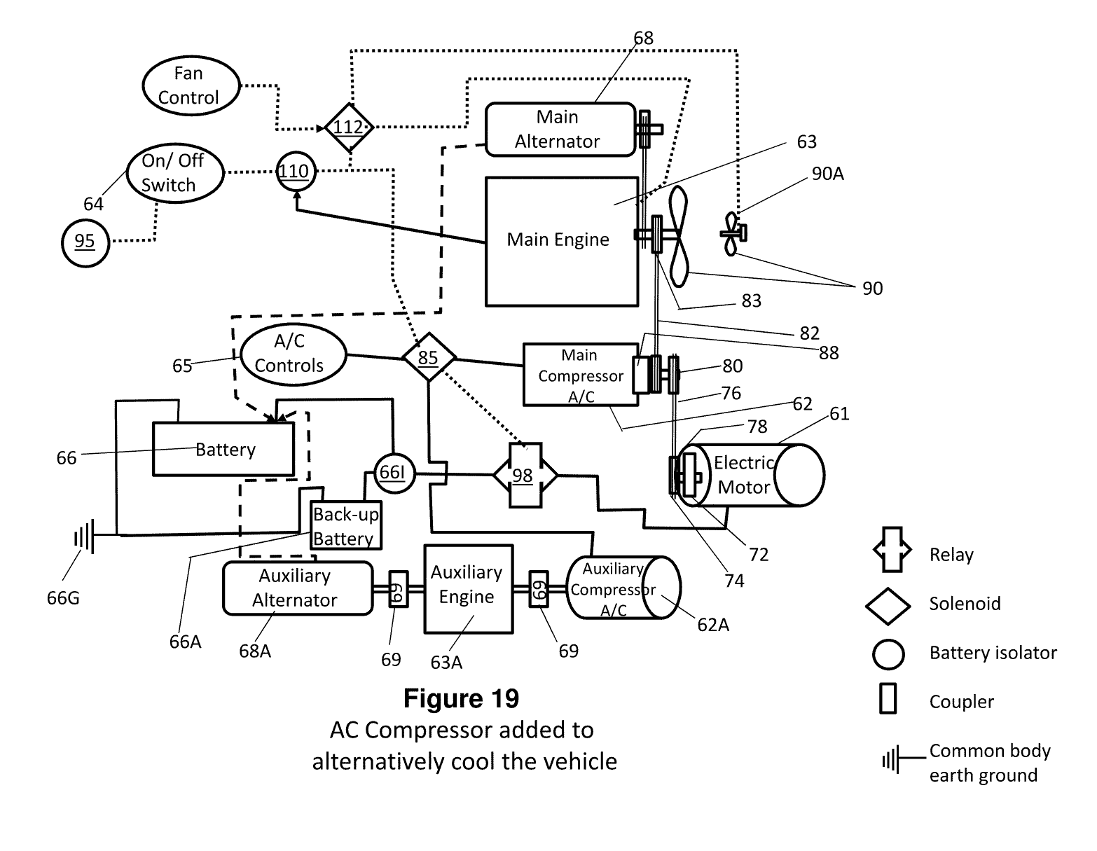

FIG. 19: AC Compressor added to alternatively cool the vehicle.

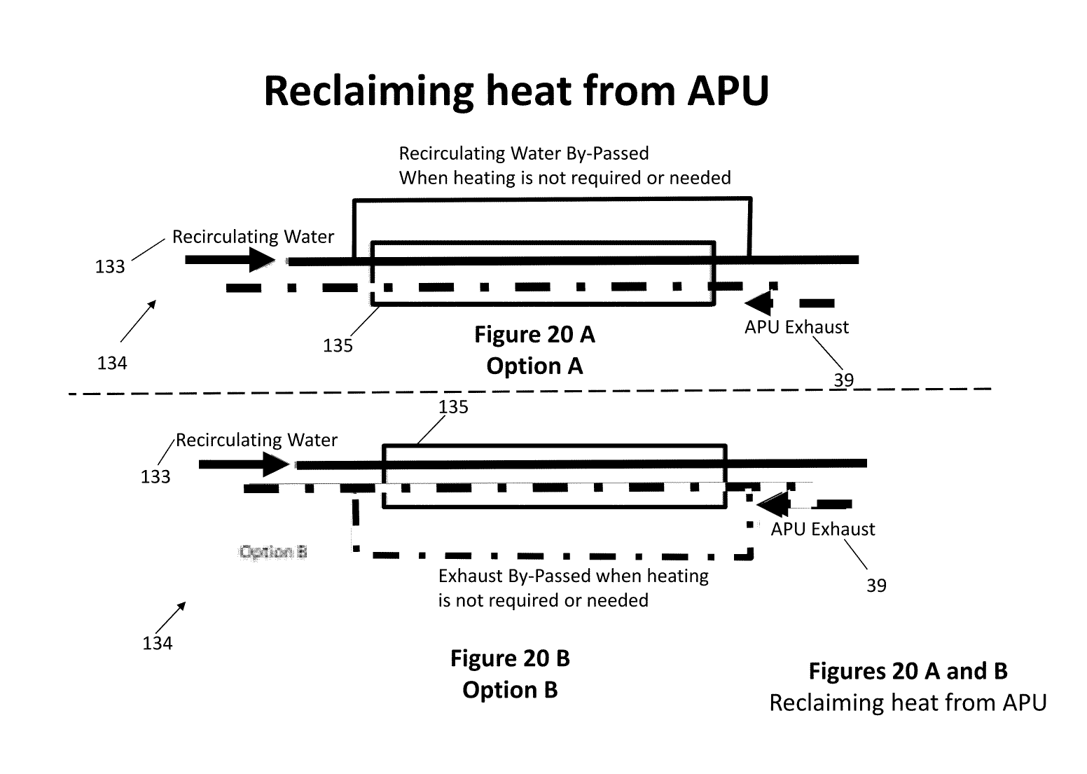

FIGS. 20 A and 20 B: Reclaiming heat from APU.

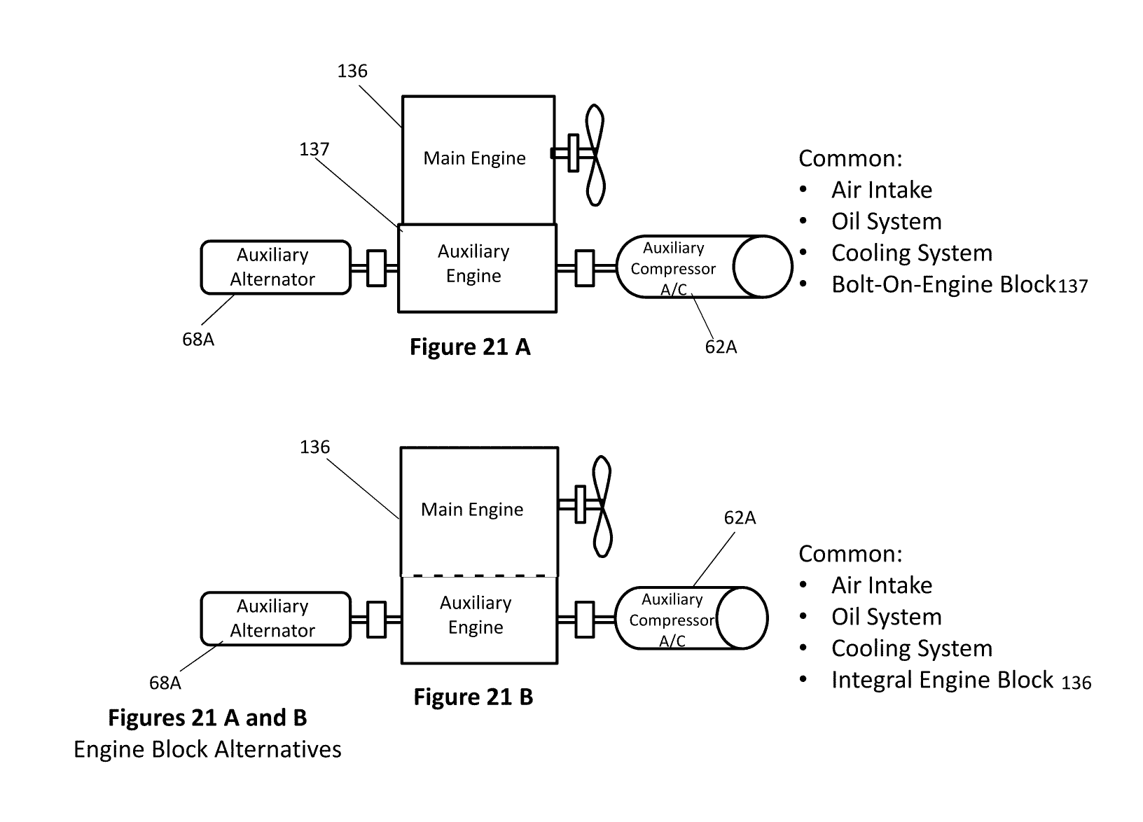

FIGS. 21 A through 21 B: Engine Block Alternatives.

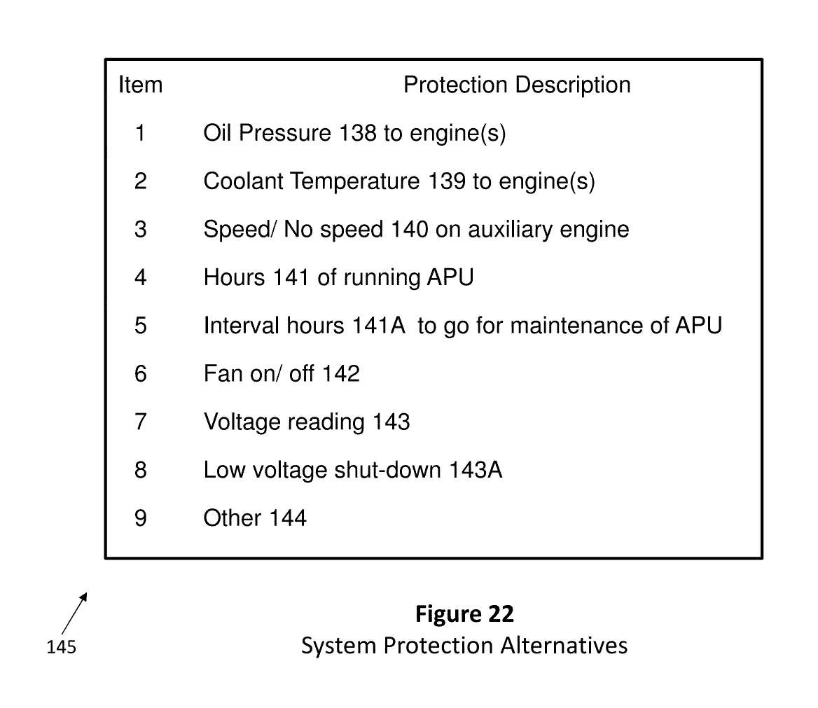

FIG. 22: System Protection Alternatives.

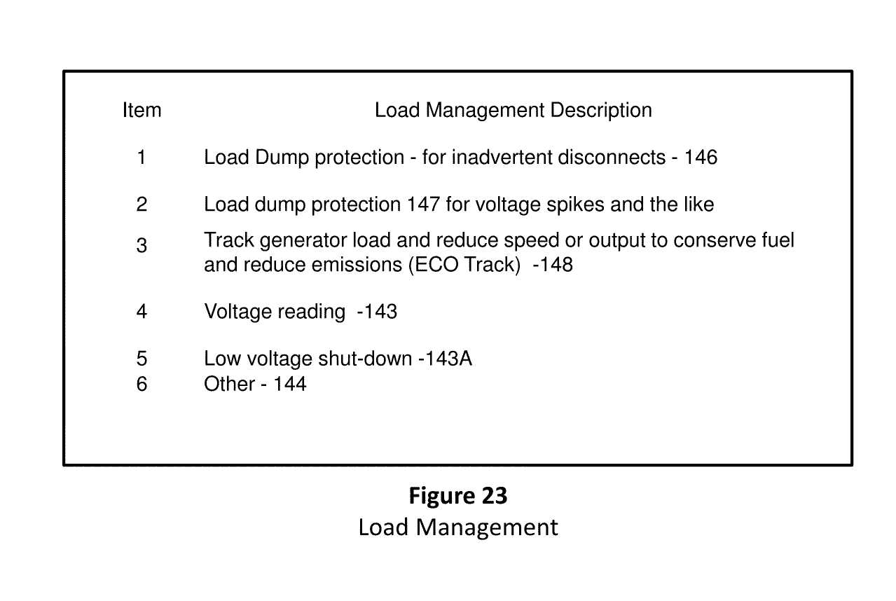

FIG. 23: Load Management.

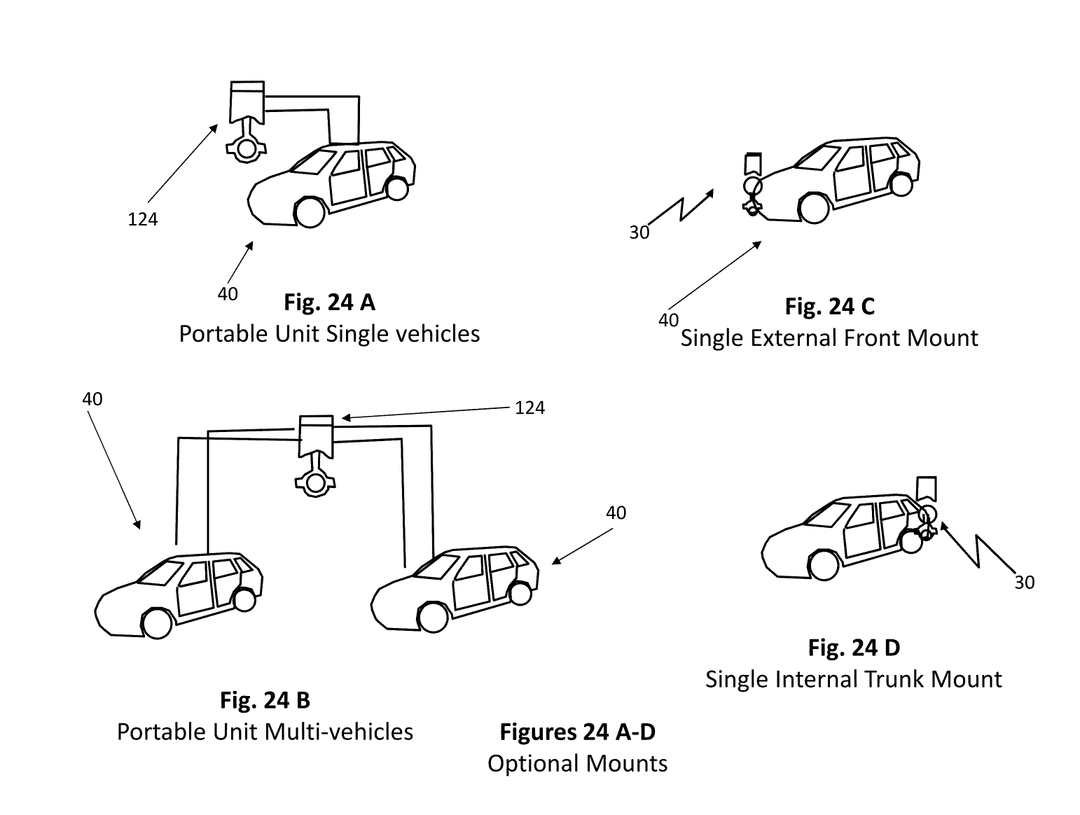

FIGS. 24 A through 24 D: Optional Mounts for D C Powerbox.

### DESCRIPTION OF THE DRAWINGS

### Reference Numerals

The following list refers to the drawings:

TABLE A
Reference numbers

| Ref # | Description |
|---|---|
| 30 | auxiliary power system called a D C Powerbox 30 |
| 31 | Fuel reservoir 31 to fuel line 56 |
| 32 | Starter motor 32 |
| 33 | Regulator 33 |
| 33A | Auxiliary regulator 33A |
| 34 | Powertrain control module 34 |
| 35 | Fusible links (Protection) 35 |
| 36 | Wiring schematic 36 |
| 37 | Wire conductor and connectors 37 |
| 38 | Charging system 38 |
| 39 | Engine 63A Exhaust system 39 |
| 40 | Vehicle with high electrical loads 40 |
| 41 | Engine compartment 41 of prototype vehicle 40 |
| 42 | Rear frame 42 |
| 43 | Rear bumper cross 43 |
| 44 | Truck area 44 of prototype vehicle 40 |
| 45 | mounting bracket 45 |
| 46 | Interior 46 of vehicle 40 |
| 47 | Dash panel 47 of vehicle 40 |
| 48 | DC power band 30 of interior controls 48 |
| 49 | Choke activation device 49 |
| 50 | Master switch 50 |
| 51 | Fuel switch 51 |
| 52 | Choke switch 52 |
| 53 | Start 53 |
| 54 | Cigarette lighter 54 |
| 55 | Amp display switch 55 |
| 55A | Amp display switch or indicator light 55A |
| 56 | Fuel line 56 |
| 57 | Fuel shut off 57 |
| 61 | Electric motor 61 |
| 62 | Main air conditioning (A/C compressor) 62 |
| 62A | Auxiliary air conditioning (A/C compressor) 62A |
| 63 | Main engine 63 |
| 63A | Auxiliary engine 63A |
| 64 | Interior vehicle switch 64 |
| 65 | A/C air conditioning control 65 |
| 66 | Main battery 66 |
| 66A | Auxiliary battery 66A |
| 66G | Common body earth ground 66G |
| 66I | Battery isolator 66I |
| 68 | Main alternator 68 |
| 68A | Auxiliary alternator 68A |
| 69 | Coupler 69 |
| 72 | Centrifugal clutch 72 |
| 74 | Pulley 74 |
| 76 | Belt 76 |
| 78 | Pulley 78 |
| 80 | Compressor drive shaft 80 |
| 82 | drive belt 82 |
| 83 | Pulley 83 |
| 85 | Relay 85 |
| 88 | Electromagnetic clutch on compressor 88 |
| 90 | Cooling fan 90 |
| 90A | Auxiliary cooling fan 90A |
| 95 | Vehicle main ignition switch 95 |
| 98 | Solenoid 98 |
| 110 | Safety shut-off relay switch 110 |
| 112 | Relay switch 112 |
| 120 | Insulated (sound and heat) trunk enclosure 120 with lid |
| | (not shown) |
| 121 | Air intake 121 for auxiliary engine 63A |
| 122 | Shunt control in schematic 122 |
| 123 | Support structure 123 for standalone unit 124 |
| 124 | Standalone unit 124 |
| 125 | Isolator 125 |
| 126 | Removable support platform 126 |
| 127 | Means for fastening 127 |
| 128 | Vehicle support 128 |
| 129 | Platform structure 129 |
| 130 | Rear vehicle side panel 130 (at wheel well) |
| 131 | Muffler/exhaust 131 of vehicle 40 |
| 132 | Measuring device 132 |
| 133 | Recirculating water 133 |
| 134 | Reclaiming heat system 134 |
| 135 | Heat exchanger 135 |
| 136 | Integral block 136 (cast at one process) |
| 137 | Auxiliary block 137 (bolt on or the like) |
| 138 | Oil pressure 138 |
| 139 | Coolant temperature 139 |
| 140 | Speed (RPM) 140 on auxiliary engine 63A |
| 141 | Hours 141 of APV 30 running |
| 141A | Interval hours 141A prior to maintenance |
| 142 | Fan on/off 142 |
| 143 | Voltage reading 143 |
| 143A | Low voltage shutdown 143A |
| 144 | Other indicators (engine, compressor, alternator |
| | conditions)144 |
| 145 | Protection system 145 anticipated |
| 146 | Load dump protection 146 |
| 147 | Voltage spike suppressors 147 |
| 148 | Economy controls and environmental controls 148 |

### DETAILED DESCRIPTION OF PREFERRED EMBODIMENT

The present development is an auxiliary power system called a D C Powerbox. This invention relates to an auxiliary engine and alternator system which will provide additional electrical power to a vehicle in lieu of the idling the primary engine. Therefore this is an auxiliary power system for use on transport vehicles powered by internal combustion engines to provide electrical power during periods when the transport vehicle's main engine can or must be shut off. Problems addressed and solved include: (a) how to reduce fuel consumption without impacting police service and (b) how to minimize the wear factor by minimizing the idle hours of a vehicle.

Emergency vehicles are use in a wide range of situations; however, police vehicles stand out as one of the most important and recognizable vehicles. Police vehicles are purpose built with many features and options. One vital requirement is a high performance engine, which enables the police to rapidly respond to critical situations and provides an advantage in high speed pursuits. An additional vital feature is a high output alternator, which typically has 75% more power capacity as compared to normal vehicles and is required to power the vehicle's electrical system, warning lights being the highest demand of this electrical power. However the power required needed to power the electrical systems is a fraction of the power supply from the high performance engine. Thus when a police vehicle is stationary with warning lights operating, the vehicle engine needs to be operating, however there is a significant amount of fuel used (wasted) due to the parasitic loads of the larger idling engine, but this is the condition of existing vehicle's design.

There is shown in FIGS. 1-23 a description and operative embodiment of an auxiliary power system called a D C Powerbox. In the drawings and illustrations, one notes well that the FIGS. 1-23 demonstrate the general configuration and alternative embodiments as examples but not limitations of the auxiliary power system called a D C Powerbox 30. The various example uses are in the operation and use section, below.

The accompanying drawings, which are incorporated in and constitute a part of this specification, illustrate an embodiment of the auxiliary power system called a D C Powerbox 30 that is preferred. The drawings together with the summary description given above and a detailed description given below serve to explain the principles of the auxiliary power system called a D C Powerbox 30.

The preferred embodiment is an auxiliary power system called a D C Powerbox comprised of: (a) an engine with an output drive shaft; (b) an alternator; (c) a means for the engine to mechanically power or drive the alternator; (d) a means for fueling the engine; (e) a means for controlling the means for fueling the engine; (f) a means for providing power from the alternator to a battery; and (g) a means to structurally mount the engine, the alternator and to removably connect the means (c), (d), (e), and (f) together as an auxiliary power system. Alternative embodiments are: The system described as a preferred embodiment wherein the means to structurally mount is to an existing police vehicle OEM or packaged system on a push bar, bracket arrangement. The system described as a preferred embodiment wherein the means to structurally mount is a standalone palletized frame. The system described as a preferred embodiment further comprised a means for providing electrical power for existing or complementary HVAC systems or as a remote stand-alone unit providing the same. The system described as a preferred embodiment further comprised of a means of recharging the vehicles primary battery requirements for normal operation as well as any secondary power requirements. The system described as a preferred embodiment wherein the means for fueling the engine is using an economically viable fuel source. The system described as a preferred embodiment further comprised of a means of displaying and monitoring aspects of charge requirements. The system described as a preferred embodiment further comprised of a means of incorporating design aspects for fuel shut off, choke, remote starting, and enabling switches. The means of providing electrical power so the vehicle can operate without using the primary engine to decrease: fuel consumption, emissions, and hours spent idling.

Some examples of the benefits for such a system are: significant fuel savings, increased in-service life of the vehicle, and lowering CO2 emissions.

FIG. 1 shows the DC Auxiliary Powerbox general arrangement 30. FIG. 1 is an image of the invention DC Powerbox 30 which is an arrangement of an engine 63A, alternator 68A and coupling 69. The engine 63A is a simple single cylinder respiration engine or equal. The coupling 69 is a typical industrial coupling, however there other options of drive between the engine and alternator, as an example: belt, direct drive gears, gearbox and etc. The alternator 63A is sized to provide an equal amount of electrical power as per the main engine alternator 68.

The prototype auxiliary engine 63A shown in FIG. 1 is a Briggs and Stratton Vanguard horizontal shaft engine. The auxiliary engine 63A is rated essentially for 6.5 HP which provides a roughly a power margin (over-powered) of 2. The auxiliary engine 63A has an electric and manual pull start along with its own intake, fuel, and exhaust 39 standalone systems; however these systems can be incorporated into the vehicle's 40 main intake, fuel, and exhaust systems for the main engine 63. The exemplary but not limiting interconnecting drive is a Lovejoy L095 coupling 69 which transfers the shaft power of the auxiliary engine 63A to the shaft of the auxiliary alternator 68A. However the coupling 69 is only one of many options drives. The coupling 69 also helps minimize torsional vibration between the auxiliary engine 63A and auxiliary alternator 68A and compensates for alignment variations between the auxiliary alternator and auxiliary engine shafts. The exemplary, yet not limiting, prototype auxiliary alternator is a 200 amp rated Delco Remy SI28 alternator, similar to other alternator the Delco Remy SI28 provides DC power. The power produced from the auxiliary alternator is regulated within the auxiliary alternator with respect to the DC voltage of the charging current by an internal auxiliary regulator 33A.

FIG. 2 is an example of vehicle 40 with high electrical loads electrical charging schematic 36. FIG. 2 is the charging system wiring schematic 36 of a vehicle 40 with high electrical needs such as an exemplary prototype vehicle like, but not limited to, a 2008 Crown Victoria police car or the like charging system. This system typical to other charging systems and in general is made up an alternator 68, regulator 33, battery 66, wire interconnections 37, and fuses/fusible links 35. However for this example of a vehicle 40 with high electrical needs the amp rating of the alternator is 200 amps and the two fuses are 175 amps each, this amperage is roughly 50% to 75% higher than comparable vehicles. This higher amperage is driven by need specific of the vehicle 40.

As one skilled in vehicle electrical charging systems can appreciate, the alternator is a generator that produces alternating current (AC) similar to the electrical current used in a residential home or offices. However in a vehicle, this AC is immediately converted to Direct Current (DC) inside the main alternator 68 by a rectifier bridge to DC. A rectifier is an electrical device that converts alternating current (AC), which periodically reverses direction, to direct current (DC), which flows in only one direction. The process is known as rectification. Physically, rectifiers take a number of forms, including vacuum tube diodes, mercury-arc valves, copper and selenium oxide rectifiers, semiconductor diodes, silicon-controlled rectifiers and other silicon-based semiconductor switches. Historically, even synchronous electromechanical switches and motors have been used Semiconductor diodes, silicon-controlled rectifiers and other silicon-based semiconductor type rectifiers are typical for vehicles.

A 12 volt DC electrical system is essentially the standard for most modern automobiles. The alternator also maintains the voltage of the charging system to between 13.5 and 14.5 volts. It does this by providing electrical power to the charging system 38. The battery 66 is charged from the alternator 68 and stores power which is used provide a stable electrical power supply when the vehicle 40 is running, and to provide electrical power for starting of the vehicle's engine 63. The interconnecting wiring 37 and fuses 35 are used to enable the flow of electrical power and the fuses are a failsafe against over loading the power system in the case of failure of the voltage regulator 33 or electrical shorts.

FIG. 3 demonstrates a DC Powerbox 30 and the Vehicle 40 with high electrical loads and the electrical charging schematic 36. Shown here is a schematic displaying: auxiliary power system called a D C Powerbox 30; auxiliary regulator 33A; charging system 38; main alternator 68; and auxiliary alternator 68A.

FIG. 4 provides a vehicle 40 with high electrical loads—“Police Package” demonstration vehicle. Shown are the vehicle 40 and the engine compartment 41 of prototype vehicle 40.

FIG. 5 is an image of a trunk 44 prior to installation of the auxiliary power system called a D C Powerbox 30. Shown and demonstrated here are: the vehicle with high electrical loads 40 and the trunk area 44 of prototype vehicle 40.

FIG. 6 demonstrates the various installed switches in the interior 46 of the vehicle 40 front left to right: master switch, fuel switch, choke switch, start, (cigarette lighter), and amp display switch. Demonstrated in this drawing are: Installed switches front left to right: master switch 50, fuel switch 51, choke switch 52, starter 53, cigarette lighter 54, and amp display switch 55.

FIG. 7 is an amp display 55A on console of the dash panel 47 mounted above trunk switch-display: a 3½″ digital blue LED DC 200 amp current meter using a 5V (2 Watt) isolated power module for a 12 V system.

FIG. 8 is an image of a DC Power box 30 installed on right rear frame 42 external to trunk 44. The view is looking forward. Here is a bracket: steel plate 45 with two (2) mounting brackets attached to frame 42, custom fabricated (insulated) steel box 120.

FIG. 9 demonstrates a fuel shut off valve 57 mounted on rear bumper cross member 43—1 Amp fuel shut off valve 57 (afcvalves.com) or equal.

FIG. 10 exhibits an intake of the auxiliary power system called a D C Powerbox 30 rotated 90 degrees CCW for installation, exhaust 39 repositioned to provide optimal heat dissipation. Here the air intake is rotated ninety (90) degrees CCW for installation, exhaust repositioned to provide optimal heat dissipation.

FIG. 11 demonstrates an installation view within thermal/sound barrier case 120 (lid not shown). This view is with the front of vehicle to the left, right side of vehicle on top. Well demonstrated are the system 30 components of auxiliary power system called a D C Powerbox 30: engine 63A; the exhaust system 39; the auxiliary alternator 68A; the coupler 69; and the insulated (sound and heat) trunk enclosure 120 with lid (not shown).

FIG. 12 shows a choke activation 49 using OEM door lock mechanism. Shown here is a configuration of the auxiliary engine 63A; auxiliary alternator 68A; exhaust system 39; and the choke activation device 49. The switch 52 is in the interior—FIG. 6.

FIG. 13 shows the image of a prototype one gallon (50 U.S. states compliant) fuel tank 31 installed on left rear of vehicle 40 providing 8 hours of operation. The tank: custom welded 50-state legal cap, brackets, brass fitting (on/off), and tubing. Also shown are the fuel line 56, fuel shut off 57, and insulated (sound and heat) trunk enclosure 120 with lid (not shown).

FIG. 14 exhibits the prototype of the wiring and harness 37 that shows shunt 122 used to monitor DC power requirements of the auxiliary power system called a D C Powerbox 30. Shown in this image are the wiring and harnesses for: master 50, fuel 51 (1 amp fuse), choke 52 (X amp fuse), start 53, hour meter 55, power from battery(ies) 66, 66A are all shown.

FIG. 15 is a sketch demonstrating a standalone unit 124—tubing, plate, dual alternators (or optionally an A/C compressor), pulleys, drive and transfer belts, engine, battery, wiring. Shown are: auxiliary engine 63A; auxiliary alternator 68A; coupler 69; support structure 123 for standalone unit 124; and standalone unit 124.

FIG. 16 is a sketch of one interface design. Wiring and harness: master, fuel (1 amp fuse), choke (X amp fuse), start, hour meter, power from battery; shunt: 200 Amp 75 mV DC shunt. Demonstrated here are: DC power band 30 of interior controls 48; main alternator 68; auxiliary alternator 68A; main battery 66; common body earth ground 66G; shunt control in schematic 122; and an isolator 125.

FIGS. 17A through 17D provide an anticipated location of the Powerbox system 30 on another police demonstration vehicle 40. Here are demonstrated, for example and not as a limitation, a removable support platform 126; a means for fastening 127; a vehicle support 128; a platform structure 129; a rear vehicle side panel 130 (at wheel well); a muffler/exhaust 131 of vehicle 40; and a measuring device 132.

FIG. 18 shows an alternator right above the A/C compressor 62—it is anticipated as an alternative application of the Powerbox system 30 mounted at this location. Shown here is the main alternator 68; the new auxiliary system can be placed in and around this engine compartment area.

FIG. 19 is a block diagram of an auxiliary A/C compressor 62A and other components potentially added to alternatively cool the vehicle 40 and charging system 38. Shown here are several components including as an example, but not limited to, an electric motor 61; a main air conditioning (A/C compressor) 62; an auxiliary air conditioning (A/C compressor) 62A; a main engine 63; an auxiliary engine 63A; an interior vehicle switch 64; an A/C air conditioning control 65; a Main battery 66; an auxiliary battery 66A; a common body earth ground 66G; a battery isolator 661; a main alternator 68; an auxiliary alternator 68A; a coupler 69; a centrifugal clutch 72; a pulley 74; a belt 76; a pulley 78; a compressor drive shaft 80; a drive belt 82; a pulley 83; a relay 85; an electromagnetic clutch on compressor 88; a cooling fan 90; an auxiliary cooling fan 90A; a vehicle main ignition switch 95; a solenoid 98; a safety shut-off relay switch 110; and a relay switch 112. The A/C compressor is driven from APU 30. Here the auxiliary engine 63A drives an electric motor 61. The auxiliary power system called a DC Powerbox 30 and a mechanical/electrical means (clutch) 88 where the A/C compressor 62A arrangement is an added option and alternative embodiment. One notes the auxiliary power system called a DC Powerbox 30 would utilize the electrical power provided by the vehicle with a backup auxiliary battery 66A (motorcycle) as a backup battery source to the APU and system. An independent battery source 66A is capable of starting the APU 30 but isolated from main vehicle battery 66 system allowing the APU 30 to be activated independently of the main vehicle battery 66 readiness and charge level.

FIGS. 20 A and 20 B: Reclaiming heat from APU portray optional reclaiming heat from the inventive APU 30. Here is shown the Engine 63A Exhaust system 39 recovering energy and heat from the recirculating water 133 along with the reclaiming heat system 134 and heat exchanger 135. One embodiment of this reclaiming 134 is electric heating system for colder climates. The device 134 recirculates heat 133 from APU 30 or generates power electricity for an electric resistance heater. It is possible APU heating/heat reclaiming systems could range from electrical resistance heating systems, to heat reclaiming systems 134.

The electrical resistance system would basically be an electrical resistance heater located within the vehicle HVAC system. This could include but not limited to vehicle engine block, vehicle radiator, and vehicle interior heat exchanger. Any of the electrical resistance heating systems could include a recirculating system 133 which would distribute heat to the entire vehicle HVAC and/or cooling systems. This system would help insure against damage to the vehicle due to extreme cold, preheat the vehicle to allowing the vehicle to be quickly deployed without thermal shock, and lastly the system could also provide comfort heating to the vehicle interior. In the case of reclaiming systems, heat from the APU would be exchanged to the to the vehicle's cooling system.

Any reclaiming systems could be automatically or manually controlled. Automatic systems would be controlled by predetermined minimum and maximum set points within the vehicle heating/cooling systems or as selected by the operator of the vehicle. A heat reclaiming a system could include a minimum and maximum set point of the vehicle engine's cooling jacket. In this example the minimum set point could be approximately 120° F. and the maximum set point could be approximately 210° F. and reclaiming system would exchange heat whenever the vehicle cooling system was below approximately 120° F. and would stop exchanging heat whenever the vehicle cooling system is above approximately 210° F. Alternatively reclaiming heat from the APU could be an option which would be engage by the operator from within the vehicle. Typically this would be comfort heating, and would be controlled by the operator desires; however the reclaiming would have over temperature fail safes.

Reclaiming heat from the APU could be done by providing a water jacket on the APU exhaust system. The recirculation of the jacket water could be from free convection or by forced convection such as a pump. The pump would powered by the APU electrical system. During times when heating of the vehicle interior is not desirable heat will not be reclaimed from the APU exhaust by either diverting the jacketed water and of the APU exhaust.

Another heat reclaiming system from the APU is capturing the heat off of the alternator rectifier heat fins. In this case the alternator could have rectifiers in parallel, and the amount of current passing through the two rectifiers would be determined to the needed heat needed to be reclaimed. In the case where no reclaiming heat is desired all of the current from the alternator would pass through the non-heat reclaiming rectifier. Alternatively, when heat is needed the current from the alternator would pass through the heat reclaiming rectifier. The control of the current passing through each rectifier could be performed automatically as previously stated or manually by the vehicle's operator as previously stated. The rejection of the heat from heat reclaiming rectifier could be exchanged to any of the vehicle systems as previously stated.

Other heat reclaiming systems 134 can be utilized: Operation in cold environments having the ability to maintain the engine in a preheated state (engine block heater) as well as any utilization of the vehicles radiator for maintaining temperature for operation or cabin heat requirements; these functions may be provided by our invention. This would mimic the same function without having to plug in to an AC outlet when operating in a remote location. Reclamation of heat form converting from AC to DC—see above. Exhaust heat of APU can be recovered. Reclaiming any heat can be made available from exhaust of the auxiliary power system called a D C Powerbox 30.

FIG. 21 provides some engine block alternatives. Here is demonstrated the auxiliary air conditioning (A/C compressor) 62A with an auxiliary alternator 68A. The shown alternative are Options (A) 136, 137 (bolt-on casting) and Option (B) 136 showing Integral block 136 (cast at one process) and auxiliary block 137 (bolt on or the like). More importantly is the option to originally design the engine [at the original OEM stage] to accept the APU 30 and combine such with the OEM engine. Those founded in the art of engine block and accessories well appreciate the desire/and or desire to design considering the electrical energy boost system offered by the auxiliary alternator 68A.

A police vehicle is built from a special purpose design:
OEM Engines are designed with built-in APU versus add on features. The vehicle needs to be available immediately when the operator is called. So another added feature is having the police vehicles as special purpose, the vehicle are equipped with high output engines for presume and high rated alternators to support the electrical demands of police equipment. And thus the APU could be incorporated with in within a vehicle design where the manufacture of the vehicle provides a space claim for an APU. Note this is not limited to an engine bolt-on configuration. An original combined block could also incorporate the APU. In addition, the Air intake of APU 30 can be compatible or part of an existing, common air intake with respect to what is present on the existing vehicle. This will mainly depend on the location of the APU—See item #3 the engine of the APU is incorporated in the vehicle engine such as a bolt on added block. In addition, the Oil system of APU may be compatible or part of an existing common oil system with primary engine. One can use the main vehicle oil system to as part of the APU oil system eliminating a separate maintenance schedule. In this option the common oil would held at an elevated temperature thus maintaining the readiness of the vehicle main engine. Finally, the Engine Block of APU may be compatible or part of existing Engine block and is incorporated with the main engine block. This is versus a separate standalone system. [Incorporating the auxiliary engine into the main engine block is likely an expensive undertaking. However, it is anticipated that the three-dimensional printing with metals or another system can make this economically feasible in the not too distant future]. If so, an added benefit is the APU engine incorporated into main engine block could drive the main vehicle alternator and AC compressor via a clutch system.

FIG. 22 is a list showing system protection alternatives 145. Shown here are:
1) Oil pressure 138 to engine(s);
2) Coolant temperature 139 to engine(s);
3) Speed/no speed 140 on auxiliary engine′
4) Hours 141 of running APU;
5) Interval hours 141A to go for maintenance of APU;
6) Fan on/off 142;
7) Voltage reading 143;
8) Low voltage shut-down 143A; and
9) Other 144.
   Protection of the APU is for example, a low engine oil/non-start feature currently installed on typical Briggs & Stratton Vanguard series. The auxiliary power system called a D C Powerbox 30 could have an oil indicator for getting this low oil condition known ahead of time. The APU 30 also includes a system which indicates maintenance concerns, alarm conditions, which could compromise the APU, and shut down or non-start for the conditions that would damage the APU. An example of a shutdown or non-start conditions would be a low oil condition. The indications of maintenance and alarm conditions can be connected and communicate to running hours and or engine loading. It is anticipated that the auxiliary power system called a D C Powerbox 30 system can have the additional safety feature where the system 30 will shut down or have a non-start for protection of the system 30.

FIG. 23 are expected functions of load management options to be available with the auxiliary power system 30 called a D C Powerbox. Here are demonstrated:

load dump protection—for inadvertent disconnects—146; load dump protection 147 for voltage spikes and the like; track generator load and reduce speed or output to conserve fuel and reduce emissions (ECO Track)—148; voltage reading—143; low voltage shut-down—143A; and other—144.

FIG. 24 are shown below in operations.

As one skilled in vehicle electrical charging systems can appreciate, these functions can operate as follows: Power adjust for load present—an ECO setting that lowers the rpm/speed of the APU 30 when a full load from the generator is not required; with incorporation of a shutdown feature when a full charge is obtained or for starting when the voltage requirement is needed. APU engine RPM/speed is adjusted to electrical demands thus further improving fuel savings; Mechanical load removal at start—the APU engine that can be uncoupled from the alternator during starting, i.e. by use of a speed clutch or the like; Electrical Load ramp—here the current form APU is ramped up slowly, i.e. capacitor or stepping up relays; and Other regulator functions—provided to protect against voltage spikes which put the vehicle's electronics at risk. This could include controlling the electrical power from the APU so that the power will ramp up at start and ramp down during shut down.

Other Considerations Include:

A. Optimization of air/fuel The APU Engine choke system that can include automatic via thermal switch, electromechanical by way of a direct operator interface, or by strictly mechanical operation. And

B. APU fuel options including fuel sources that could be natural gas, propane, or hydrogen.

The details mentioned here are exemplary and not limiting. Other specific process, methods and manners specific to this auxiliary power system called a D C Powerbox 30 as described by the embodiments of the auxiliary power system 30 can be added as a person having ordinary skill in the field of auxiliary power systems, apparatuses and methods and their uses well appreciates.

### Operation of the Preferred Embodiment

The auxiliary power system called a D C Powerbox system 30 has been described in the above embodiment. The manner of how the device operates is described below. The operation of the invention DC Powerbox 30 is a seamless transition of electrical power, which maintains the readiness/operation of the vehicle 40 without the need to idle the vehicle's main engine 63. The invention DC Powerbox 30 can be deployed when the police officer would anticipate that the police vehicle 40 will be stationary but would still need electrical power. Such in the cases of crowd control, accident scenes, standoffs and etc.

The invention DC Powerbox 30 would be deployed by the following:
1) The vehicle is switched to park
2) The invention DC Powerbox is activated by the police officer by a switch 50 within the interior 46 of the vehicle 40.
3) The engine 63A of the invention DC Powerbox 30 is started again by the officer activating a start switch 53 within the vehicle 40.
4) Once the invention DC Powerbox 30 is generating electrical power there is feedback to the officer, i.e. an indicator light, display 55A or a similar notification.
5) The officer then can stop the engine by switching the vehicle 40 ignition switch 35 to accessory.
6) The invention DC Powerbox continues to generate electrical power until the vehicle is switched back over to its primary system.
7) Once the primary system is operating the invention DC

Powerbox can be deactivated.

As noted the electrical power from the invention DC Powerbox will provided all of the electrical power required by the vehicle when the primary engine is not being used, however modifications to the existing heating and cooling systems could be powered by the invention DC Powerbox in lieu of power from the primary engine.

Many uses are anticipated for the auxiliary power system called a D C Powerbox 30. Some examples, and not limitations, are shown in the following Table:

| Item | Description of Use |
|---|---|
| 1 | Police Vehicles |
| 2 | Fire Vehicles |
| 3 | Ambulance and Emergency Vehicles |
| 4 | Military Vehicles |
| 5 | Homeland Security Vehicles including Border Patrol |
| | Vehicles |
| 6 | Search and Rescue Vehicles |

There are several embodiments anticipated: Prototype System of the auxiliary power system called a D C Powerbox 30—The following describes the system as developed by DC Powerbox for validating the original design, which is described in this application. The system uses a Briggs & Stratton 6.5 hp electric start gasoline engine 63A coupled to a Delco Remy 180 amp alternator 68A. The Engine 63A has been modified first with the addition of a separate fuel system comprised of an additional fuel tank 31 mounted in the trunk 44. The 1 gallon fuel tank 31 (which was shown to provide sufficient electrical power for up to eight hours of operation on 1 gallon of fuel) fitted with a CARB compliant cap [which allows operation in all 50 states], as well as a manual shut off valve 57 just below the tank 31. The fuel line 56 then goes to another fuel shutoff valve 56 provided by AFC technologies with a 1 amp fuse recommended by the manufacturer and which is controlled from within the cabin (fuel shutoff switch 51) of the vehicle during operation. The actual control and use of the device is from inside the vehicle includes: a remote ground or enable switch for arming or disarming the system 50, a fuel on/off switch 51, a choke switch 52 which uses a spare door lock 49 (similar to what is currently installed on the vehicle) for applying choke during start up, a push button start switch 53, and an additional switch 55 to activate a display 55A which shows the amount of amps currently being used by the vehicle and its accessories.

The operator, while the vehicle is idling, will determine whether and when to activate the system based on the extended time expected or required to remain in the current location (for whatever reason); the operation of the accessories complemented by the vehicles HVAC system or other remote devices located within the vehicle as well. As described below, the operation of the system will take place by: arming the system, turning the fuel on, applying choke as required, starting the engine, monitoring the required amps currently being used by the various electrical and HVAC requirements of the system, and then turning the vehicles' primary engine off which provides the basis of reducing wear on the vehicles primary engine, as well as fuel savings, and reducing CO2 emissions.

The prototype system was developed, tested, and was installed within the trunk of a 2008 Ford Crown Victoria 40. Subsequent empirical testing will be implemented on other police and emergency vehicles in production utilizing modifications on either the front push bumper, or in the rear of the vehicle on a removable connection mechanism. Trailer hitches may be utilized for some mountings but are strictly forbidden with police vehicles due to chase constraints and regulations. Another concept in development is to produce a stand-alone unit 124 that is mobile or can be deployed on scene or modular depending on the vehicles mission requirements.

FIGS. 24 A through 24 D portray optional mounts for D C Powerbox. In FIG. 24 A is a portable device 124 with a single vehicle 40. Here one sees that Portable 124 works with an individual vehicle 40, has inter connection, self-contained—cooling, fuel, starting system, and additional features/options such as electrical heating of main vehicle and electric driven AC compressor. In FIG. 24 B is a portable device 124 with a multiple vehicles 40. Here one sees portable multiple vehicles 40, inter connection, self-contained—cooling, fuel, starting system, and additional features/options such as electrical heating of main vehicle and electric driven AC compressor. In FIG. 24 C is an external device 30 with a single vehicle 40. Here is demonstrated external vehicle mounting, inter connection, self-Contained—cooling, fuel, starting system, and additional features/options—change RPM of APU Engine to match demand, electrical heating of main vehicle, electric driven AC compressor, and switch to efficient mode—auto on/off of APU and main engine. In FIG. 24 D is an internal device 30 with a single vehicle 40. Here is shown Internal to Vehicle the inter connection, self-Contained—cooling, fuel, starting system, and additional features/options—change RPM of APU Engine to match demand, electrical heating of main vehicle, electric driven AC compressor, switch to efficient mode—auto on/off of APU and main engine, heat recovery systems, exhaust, water jacket, common exhaust and common fuel system with the vehicle.

Police enforcement varies greatly from crowd control to high speed pursuits. The dynamics of police enforcement is further increased by the municipalities they serve. Typically municipalities will have similar police enforcement requirements such as: traffic control, cording off areas after natural or man-made disasters and responding to criminal activities. The larger metropolitan cities will typically have a higher need for crowd control due to: sporting events, parades, festivals, celebrations and etc. The smaller municipalities will also have the need for crowd control but at an obviously lower requirement. Move over larger municipalities may have designated police officer and vehicles responsible for crowd control. In this situation a reserve officer would be assigned crowd control while more season officers would be assigned the regular duties. Additionally, because of the advantages of K-9 units many municipalities have dedicated K-9 police vehicles. Thus there are numerous situations that police vehicle are idled.

The objective in using the APU provided by DC Powerbox is to decrease fuel consumption while the police vehicle idles due to the variety of tasks perform by police departments. DC Powerbox has five major configurations: portable single vehicle, portable multi vehicle, an external vehicle mounted configuration, an internal mounted configuration and a configuration which is incorporated within the vehicle main engine compartment. Because police vehicle readiness is crucial along with climate control for K-9 units, all of the DC Powerbox configurations have an additional system which insures the readiness and the HVAC control of the vehicle. This system works in conjunction with the DC Powerbox. This system constantly monitors the status of the deployed DC Powerbox and if there is a failure of the DC Powerbox this system will indicate starting of the vehicle main engine. The failure of the DC Powerbox could be from running out of gas or a DC Powerbox fail to start condition. This system will also activate the main engine for HVAC or other vehicle readiness conditions. At one time a product was developed which started and stopped the main vehicle with respect to the voltage level of the vehicle electrical system. Thus this system would start the vehicle when the power of the battery was drained down and the restart the engine to recharge the battery. This system was only marginally successful with saving fuel because there is a significant energy lost due to parasitic load while the engine is running to recharge the depleted battery. More over because in this system attempts to start the vehicle engine once the battery is depleted there is an anticipated risk that the engine will not start due to the depleted battery. Additionally, this system did not make provisions for environmental conditions within the vehicle. (For Informational purposes only).

The portable single vehicle configuration is self-contained which includes fuel, oil, air intake, exhaust, and cooling systems; this configuration as in its name is portable and will provide the electrical power for one or more vehicles. The unit could be stored within the police vehicle and deployed at the judgment of the officer. It is anticipated the officer would know when he would expect to idle for a significant time such as crowd control before during and after major sporting events. The portable single vehicle configuration has the advantage of being switch between vehicles. This provides a flexibility of being to be deployed from different vehicles but this configuration also takes a minimum amount of time to deploy. Because of the flexibility of switching this system to different vehicles this option may be more of the interest of smaller municipalities.

Deployment of the portable single unit would consist of removing the unit from the vehicle storage, securing it to the police vehicle, connecting the harness between the vehicle and portable single unit, starting the engine of the portable unit, and powering down the main engine of the vehicle. As mentioned the portable unit is stored with in the vehicle and removed to be deployed. To insure readiness and protection of the portable unit includes a fixturing system which secures the portable unit within the vehicle. This fixturing will insure the portable unit is held in the vertical direction thus removing the concern of spilled fluids and hydraulic piston lock. Securing the portable single unit to the vehicle is done by folding down the outriggers of the portable single unit base. The outriggers are then wedge against the bottom of the vehicle's wheel (so it doesn't vibrate or crawl away during operation). After the portable single unit is secured the harness between the vehicle and portable unit is connected. This connection is provided by a junction in which snaps together providing a positive connect for the main power cables and provides controlling logic of the engine of the portable single unit and the control of the vehicle's engine.

The typical jumper cable style connection would not be suited for the amount of current that passes from the portable single unit and the main vehicle charging system, thus the design of the interconnecting harness and connection junction. The harness is in two sections one section assembled to the vehicle charging system and the other assembled to the portable single unit, and the connection junction enables the current to travel from the portable signal unit to the vehicle charging system, plus provides a signal that controls the start enabling of the portable single unit. Because it is crucial that the harness to be connected before the portable unit is started there is a contact switch with in the connection junction. Once the connection junction is assembled the contact switch is switch and the engine of the portable single unit is enabled to start. In addition to enabling the engine of the portable single unit to start the connection junction also includes a switch which communicates to the police vehicle. This switch indicates to the vehicle that it is connected to the portable single unit and will alarm and or shut the main engine down if the vehicle is put in to gear.

Again, the roles of police officers varies greatly and one of the areas that the DC Powerbox will benefit the police department is when the police office is faced with responding to un-anticipated crises which requires the officer to remain on scene for an extended period of time. Although there are many situations, the ones that most people can relate to are: traffic accidents and coordinating off areas due to natural disasters. The police officer time at a scene of an automobile accident varies greatly from minutes for a minor fender bender to hours for more serious accidents. In the cases of coordinating off areas, daily police officers are cording off areas in order to warn the public about a dangerous situation such as a flood or downed power lines. Because these are unanticipated events and it would not be an unrealistic expectation for a police officer to deploy the portable single DC Powerbox unit, and to facilitate the use of the DC Powerbox in lieu of idling the main engine, in using the semi permanently fastened DC Powerbox as previously defined. The semi permanently fastened DC Powerbox would be mounted to the exterior of the vehicle similar to or incorporated into a push bumper or bumper guard. Because it would be semi permanently mounted to the vehicle there would be an interconnecting electrical control harness. The interconnecting control harness would allow the police office to start the semi-permanent mounted DC Powerbox from within the vehicle as what was developed in the demonstrate prototype but because police work is dynamic and fluid this variation of DC Powerbox has the feature of starting automatically.

Other embodiments of the invention are possible. Although the description above contains much specificity, these should not be construed as limiting the scope of the invention, but as merely providing illustrations of some of the presently preferred embodiments of this invention. It is also contemplated that various combinations or sub-combinations of the specific features and aspects of the embodiments may be made and still fall within the scope of the inventions. It should be understood that various features and aspects of the disclosed embodiments can be combined with or substituted for one another in order to form varying modes of the disclosed inventions. Thus, it is intended that the scope of at least some of the present inventions herein disclosed should not be limited by the particular disclosed embodiments described above.

With this description it is to be understood that the auxiliary power system called a D C Powerbox 30 is not to be limited to only the disclosed embodiment of product. The features of the auxiliary power system called a D C Powerbox 30 are intended to cover various modifications and equivalent arrangements included within the spirit and scope of the description.

Unless defined otherwise, all technical and scientific terms used herein have the same meaning as commonly understood by one of ordinary skill in the art to which these inventions belong. Although any methods and materials similar or equivalent to those described herein can also be used in the practice or testing of the present inventions, the preferred methods and materials are now described. All patents and publications mentioned herein, including those cited in the Background of the application, are hereby incorporated by reference to disclose and described the methods and/or materials in connection with which the publications are cited.

The publications discussed herein are provided solely for their disclosure prior to the filing date of the present application. Nothing herein is to be construed as an admission that the present inventions are not entitled to antedate such publication by virtue of prior invention. Further, the dates of publication provided may be different from the actual publication dates which may need to be independently confirmed.

Thus the scope of this invention should be determined by the appended claims and their legal equivalents. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims.

The terms recited in the claims should be given their ordinary and customary meaning as determined by reference to relevant entries (e.g., definition of “plane” as a carpenter's tool would not be relevant to the use of the term “plane” when used to refer to an airplane, etc.) in dictionaries (e.g., widely used general reference dictionaries and/or relevant technical dictionaries), commonly understood meanings by those in the art, etc., with the understanding that the broadest meaning imparted by any one or combination of these sources should be given to the claim terms (e.g., two or more relevant dictionary entries should be combined to provide the broadest meaning of the combination of entries, etc.) subject only to the following exceptions: (a) if a term is used herein in a manner more expansive than its ordinary and customary meaning, the term should be given its ordinary and customary meaning plus the additional expansive meaning, or (b) if a term has been explicitly defined to have a different meaning by reciting the term followed by the phrase “as used herein shall mean” or similar language (e.g., “herein this term means,” “as defined herein,” “for the purposes of this disclosure [the term] shall mean,” etc.). References to specific examples, use of “i.e.,” use of the word “invention,” etc., are not meant to invoke exception (b) or otherwise restrict the scope of the recited claim terms. Other than situations where exception (b) applies, nothing contained herein should be considered a disclaimer or disavowal of claim scope. Accordingly, the subject matter recited in the claims is not coextensive with and should not be interpreted to be coextensive with any particular embodiment, feature, or combination of features shown herein. This is true even if only a single embodiment of the particular feature or combination of features is illustrated and described herein. Thus, the appended claims should be read to be given their broadest interpretation in view of the prior art and the ordinary meaning of the claim terms.

As used herein, spatial or directional terms, such as “left,” “right,” “front,” “back,” and the like, relate to the subject matter as it is shown in the drawing FIGS. However, it is to be understood that the subject matter described herein may assume various alternative orientations and, accordingly, such terms are not to be considered as limiting. Furthermore, as used herein (i.e., in the claims and the specification), articles such as “the,” “a,” and “an” can connote the singular or plural. Also, as used herein, the word “or” when used without a preceding “either” (or other similar language indicating that “or” is unequivocally meant to be exclusive—e.g., only one of x or y, etc.) shall be interpreted to be inclusive (e.g., “x or y” means one or both x or y). Likewise, as used herein, the term “and/or” shall also be interpreted to be inclusive (e.g., “x and/or y” means one or both x or y). In situations where “and/or” or “or” are used as a conjunction for a group of three or more items, the group should be interpreted to include one item alone, all of the items together, or any combination or number of the items. Moreover, terms used in the specification and claims such as have, having, include, and including should be construed to be synonymous with the terms comprise and comprising.

Unless otherwise indicated, all numbers or expressions, such as those expressing dimensions, physical characteristics, etc. used in the specification (other than the claims) are understood as modified in all instances by the term “approximately.” At the very least, and not as an attempt to limit the application of the doctrine of equivalents to the claims, each numerical parameter recited in the specification or claims which is modified by the term “approximately” should at least be construed in light of the number of recited significant digits and by applying ordinary rounding techniques.

What is claimed and desired to be secured by this patent is:

1. An auxiliary power system called a D C Powerbox comprised of:
(a) an engine with an output drive shaft;
(b) an alternator;
(c) a means for the engine to mechanically power or drive the alternator;
(d) a means for fueling the engine;
(e) a means for controlling the means for fueling the engine;
(f) a means for providing power from the alternator to a battery; and
(g) a means to structurally mount the engine, the alternator and to removably connect the means (c), (d), (e), and (f) together as an auxiliary power system.

2. The system in claim 1 wherein the means to structurally mount to an existing police vehicle OEM is a packaged system on a push bar or bracket assembly.

3. The system in claim 1 wherein the means to structurally mount is a stand-alone palletized frame.

4. The system in claim 1 further comprised of a means for providing electrical power for existing and/or complementary HVAC systems is a remote stand-alone unit providing the same.

5. The system in claim 1 further comprised of a means for recharging the vehicles primary battery requirements for a normal operation and secondary power requirements.

6. The system in claim 1 wherein the means for fueling the engine is by using an economically viable fuel source.

7. The system in claim 1 further comprised of a means for displaying and for monitoring aspects of charge requirements.

8. The system in claim 1 further comprised of a means for incorporating design aspects of fuel shut off, choke, remote starting, and enabling switches.

9. The system in claim 4 wherein the means for providing electrical power can enable the vehicle to operate absent using the primary engine and thereby enabling a decrease in fuel consumption, emissions, and hours spent idling.

10. The system in claim 4 that utilizes an inverter to deliver an optional AC power supply.

11. The system in claim 4 that has an interface mechanism with vehicles existing charging system and HVAC system as provided by OEM.

12. The system in claim 1 further comprised of an auxiliary Air Conditioning compressor which alternatively can cool the vehicle.

13. The system in claim 1 further comprised of a bypass for recirculating water and/or exhaust gasses.

14. The system in claim 1 further comprised of an engine block for a main and an auxiliary engine block configured to have common air intakes, oil lubricating systems and cooling systems.

15. The system in claim 1 further comprised of a protection system configured to monitor oil pressure; coolant temperature; speed/no speed monitoring; hours of engine run time monitoring; maintenance schedule points recording hours of the engine; fan operating condition as to on/off; voltage condition of electrical system; and low voltage shutdown.

16. The system in claim 1 further comprised of load management configurations.

17. The system in claim 8 with autonomous functions as a stand-alone system.

18. The system in claim 4 further comprised of an autonomous sensing system HVAC for a K9 unit and or a service animal, with provisions for a CO2 alarm for occupants.

19. The system in claim 4 wherein the remote standalone palletized frame is used with a single vehicle.

20. The system in claim 3 wherein the remote standalone palletized frame is used with more than one vehicle.
